(19) United States
(12) Patent Application Publication
(10) Pub. No.: US 2009/0118337 A1
(43) Pub. Date: May 7, 2009

(54) Methods and compositions for treating inflammation

(76) Inventor: Pamela B. Davis, Cleveland Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 11/916,410

(22) PCT Filed: Jun. 2, 2006
(86) PCT No.: PCT/US06/21304

§ 371 Date: May 21, 2008

Related U.S. Application Data
(60) Provisional application No. 60/687,511, filed on Jun. 3, 2005.

(51) Int. Cl. A61K 31/4439; A61K 31/426; A61K 31/421; A61K 31/41

(52) U.S. Cl. 514/342; 514/365; 514/360; 514/369; 514/376

(57) ABSTRACT

A method of treating a subject with a cystic fibrosis related disorder includes administering a therapeutically effective amount of at least one PPARγ agonist or a derivative thereof.

### RELATED APPLICATION

This application claims priority from U.S. provisional patent application Ser. No. 60/687,511, filed on Jun. 3, 2005, the subject matter of which is incorporated herein by reference.

The invention described in this application was supported, at least in part, from the National Institute of Health, and thus the United States government may have certain rights in the invention.

### FIELD OF THE INVENTION

The present invention relates to methods and compositions used for treating inflammation and particularly relates to methods and compositions for treating inflammation associated with NF-κB activation.

### BACKGROUND

Inflammation can be defined as a localized response in the body to cellular injury or infection. Inflammation can be characterized by, for example, dilation of blood vessels with increased permeability and blood flow, exudation of fluids, and leukocyte migration to the local areas with increased concentrations of cytokines.

Although an inflammatory response is often beneficial, in some cases, continued or excess inflammation may be detrimental to an individual. For example, individuals with cystic fibrosis (CF) may develop bacterial infections in the lungs. Along with the bacterial infections, a vigorous inflammatory response often develops in the lungs. This inflammatory response may become excessive and, eventually, become deleterious to the individual and even promote or facilitate the continuing bacterial infection.

Anti-inflammatory therapy has been used to treat CF individuals with excessive inflammatory responses. However, existing anti-inflammatory treatments used to treat CF individuals may cause adverse or undesirable side effects.

### SUMMARY OF THE INVENTION

The present invention relates to a method of treating a subject with a cystic fibrosis related disorder. In the method a therapeutically effective amount of at least one PPARγ agonist or a derivative thereof is administered to the subject. The PPARγ agonist or derivative thereof is administered to the subject in an amount effective to suppress airway inflammation. The PPARγ agonist or derivative thereof can also be administered at an amount effective to inhibit NF-κB activation.

In one aspect of the invention the PPARγ agonist or a derivative thereof comprises thiazolidinedione or a derivative thereof. In another aspect of the invention, the PPARγ agonist or a derivative thereof comprises at least one compound or a pharmaceutically salt thereof selected from the group consisting of (+)-5[[4-[(3,4-dihydro-6-hydroxy-2,5,7,8-tetramethyl-2H-1-benzopyran-2-yl)methoxy]phenyl]methyl]-2,4-thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4dione; 5-[4-[2-[(N-methyl-N-(phenoxycarbonyl)amino]ethoxy]benzyl]thiazolidine-2,4dione; 5-[4-[2-phenoxyethoxy)benzyl]thiazolidine-2,4-dione; 5-[4-[2-(4-chlorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

The present invention also relates to a method of treating inflammation associated with NF-κB activation in a subject. In the method, a therapeutically effective amount of at least one PPARγ agonist or a derivative thereof is administered to the subject. The inflammation can be associated with a cystic fibrosis related disorder. In an aspect of the invention, the PPARγ agonist or the derivative thereof used to treat inflammation associated with NF-κB activation can comprise a thiazolidinedione or a derivative thereof.

In another aspect of the invention, the PPARγ agonist or a derivative thereof used to treat inflammation associated with NF-κB activation can comprise at least one compound or a pharmaceutically salt thereof selected from the group consisting of (+)-5[[4-[(3,4-dihydro-6-hydroxy-2,5,7,8-tetramethyl-2H-1-benzopyran-2-yl)methoxy]phenyl]methyl]-2,4-thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4dione; 5-[4-[2-[(N-methyl-N-(phenoxycarbonyl)amino]ethoxy]benzyl]thiazolidine-2,4dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

### BRIEF DESCRIPTION OF THE DRAWINGS

FIG. 1 is a chart illustrating the amount of activated p50 in the nucleus of 16HBEo-sense and antisense cells under basal conditions (no stimulation) and under conditions of stimulation.

FIGS. 2(A-B) are charts illustrating luciferase expression in 16HBEo-sense and antisense cell transfected with constructs containing the luciferase gene driven by NF-κB (FIG. 2A) or the native IL-8 (FIG. 2B) and exposed to PAO1. Promoter activity was assessed by measuring luciferase activity.

FIG. 3 is Western blot illustrating both cytoplasmic nuclear extracts of 9HTEo- and 16HBEo cell pairs (CF phenotype and non-CF phenotype).

FIG. 4 are electrophoretic mobility shift assays (EMSA) using PPRE demonstrating that DNA binding by components of the nuclear extract from these cells lines identified the binding protein as PPARγ.

FIG. 5 illustrates that gelatin zymography shows that well-differentiated airway epithelial cells grown at air-liquid interface release MMP-9, which can digest the protein in the gel. Release of MMP-9 is also inhibited by PPARγ agonists.

FIGS. 6 and 7 are charts illustrating that when PPARγ agonists are added to well-differentiated airway epithelial cells there is significant inhibition of cytokine production (IL-8, IL-6, GM-SCF) by the agonists.

FIGS. 8 and 9 are blots of immunoprecipitation assays that illustrate PPARγ can interact directly with NF-κB. FIG. 8 illustrates that antibodies to both the p50 and the p65 subunit of NF-κB can pull down PPARγ.

FIG. 9 illustrates that antibodies to PPARγ also pulled down p50 and p65.

FIG. 10 are blots of an immunoprecipitation assay that illustrate NF-κB showing reduced interaction with PPARγ with PAO1 treatment, and in CF compared to WT.

FIG. 11 is a blot of an immunoprecipitation assay that illustrates that PPARγ agonists preserve the interaction between NF-κB and PPARγ in the face of inflammatory stimulation in CF cells.

FIGS. 12-14 are charts illustrating that CF mice treated with pioglitazone have a significant reduction in inflammatory response.

### DETAILED DESCRIPTION

As used herein, the term “therapeutically effective amount” refers to that amount of a composition that results in anelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

As used herein, the term “PPARγ agonist” refers to a compound or composition, which when combined with PPARγ, directly or indirectly stimulates or increases an in vivo or in vitro reaction typical for the receptor (e.g., transcriptional regulation activity). The increased reaction can be measured by any of a variety of assays known to those skilled in the art. An example of a PPARγ agonist is a thiazolidinedione compound, such as troglitazone, rosiglitazone, pioglitazone, ciglitazone, WAY-120,744, englitazone, AD 5075, darglitazone, and congeners, analogs, derivatives, and pharmaceutically acceptable salts thereof.

As used herein, the terms “host” and “subject” refer to any animal, including, but not limited to, humans and non-human animals (e.g., rodents, arthropods, insects, fish (e.g., zebrafish), non-human primates, ovines, bovines, ruminants, lagomorphs, porcines, caprmies, equines, canines, felines, aves, etc.), which is to be the recipient of a particular treatment. Typically, the terms “host,” “patient,” and “subject” are used interchangeably herein in reference to a human subject.

As used herein, the terms “subject suffering from cystic fibrosis”, “subject having cystic fibrosis” or “subjects identified with cystic fibrosis” refers to subjects that are identified as having or likely having a mutation in the gene that encodes cystic fibrosis transmembrane conductance regulator (CFTR) protein, which cause cystic fibrosis.

The term “biologically active,” as used herein, refers to a protein or other biologically active molecules (e.g., catalytic RNA) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists include, but are not limited to proteins, nucleic acids, carbohydrates, lipids or any other molecules which bind or interact with biologically active molecules. For example, agonists can alter the activity of gene transcription by interacting with RNA polymerase directly or through a transcription factor or signal transduction pathway.

The term “modulate,” as used herein, refers to a change in the biological activity of a biologically active molecule. Modulation can be an increase or a decrease in activity, a change in binding characteristics, or any other change in the; biological, functional, or immunological properties of biologically active molecules.

As used herein, the term “in vitro” refers to an artificial environment and to processes or reactions that occur within an artificial environment. In vitro environments consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that are used to treat or prevent a disease, illness) sickness or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing or eradicating a deleterious or harmful condition associated with CF-related disease. Examples of such treatment include: decreasing bacterial infection, increasing pulmonary function, down regulation of pro-inflammatory cytokines and upregulating mononuclear cell accumulation.

For the purposes of this application, the terms “CF-related disease(s) or disorder(s)” includes diseases and/or conditions related to Cystic Fibrosis (CF). Examples of such diseases include cystic fibrosis, variant cystic fibrosis and non-CF bronchiectasis.

The term “Cystic fibrosis (CF)” refers to an autosomal recessive disorder with a highly variable clinical presentation. Cystic fibrosis is predominantly a disorder of infants, children and young adults, in which there is widespread dysfunction of the exocrine glands, characterized by signs of chronic pulmonary disease, pancreatic deficiency, abnormally high levels of electrolytes in the sweat and occasionally by biliary cirrhosis. Also associated with the disorder is an ineffective immunologic defense against bacteria as well as dysregulated inflammation in the lungs. The classic form of cystic fibrosis is caused by loss-of-function mutations in the cystic fibrosis transmembrane conductance regulator (CFTR) gene. Nonclassic forms of cystic fibrosis have been associated with mutations that reduce but do not eliminate the function of the CFTR protein.

“Variant cystic fibrosis” is a disorder which is phenotypically indistinguishable from cystic fibrosis, but which is not associated with mutations in the CFTR gene (N Engl J Med. 2002; 347: 401-7).

The present invention relates to methods and compositions for treating cystic fibrosis related diseases or disorders. In particular, the present invention provides therapeutic agents that mitigate the production of proinflammatory products involved in cystic fibrosis related disorders. The presence of inflammatory cytokines, such as IL-8, IL-6, GM-CSF, and ICAM-1, at elevated levels have been detected in surface or media from cystic fibrosis airway epithelial cells. Human airway epithelial cells in culture with the cystic fibrosis phenotype usually can invariably produce more inflammatory cytokines in response to P. aertiginosa (PAO1A) or TNF-α plus IL-1β. This increase in proinflammatory mediator production can be associated with increased activation of NF-κB as well as an increase in activation of other transcription factors.

The compositions and methods of the present invention are based on the use of PPARγ agonists to suppress, inhibit, or mitigate a diverse range of inflammatory responses associated with cystic fibrosis related disorders. These inflammatory responses can be associated of NF-κB mediated or driven process or other proinflammatory processes. PPARγ agonists in accordance with the present invention can be administered to a subject being treated along with or prior to inflammatory stimuli to inhibit NF-κB driven processes, including the production of IL-8, IL-6, and GM-CSF and the release of matrix metalloproteinase 9 (MMP9) in response to pseudomonas or cytokine stimulation. The present invention therefore shows that PPARγ agonists can exert at least a portion of their activity at the level of gene transcription.

Although it is not necessary to understand the mechanisms in order to practice the present invention, and it is not intended that the present invention be so limited, it is shown by the present invention that PPARγ interacts with proinflammatory transcription factors, such as NF-κB, to prevent their function and that under inflammatory stimulation associated with cystic fibrosis related disorders this PPARγ interaction is reduced. It is contemplated that binding of PPARγ agonists in accordance with the present invention can protect PPARγ from post translational modification or change its conformation so that under inflammatory stimulation PPARγ can still interact with transcription factors, such as NF-κB.

One aspect of the present invention relates to method of treating a cystic fibrosis related disorder in a subject by administering a therapeutically effective amount of compounds that include PPARγ agonists or therapeutically effective derivatives thereof to the subject to regulate the production of proinflammatory products involved in cystic fibrosis related disorders. In one aspect of the invention the PPARγ agonists can include, for example, prostaglandin J2 (PGJ2) and analogs thereof (e.g., A2-prostaglandin J2 and 15-deoxy-2 4prostaglandin J2), members of the prostaglandin D2 family of compounds, docosahexaenoic acid (DHA), and thiazolidinediones (e.g., ciglitazone, troglitazone, pioglitazone, and rosiglitazone).

In addition, such agents include, but are not limited to, L tyrosinie based compounds, farglitazar, GW7845, indole-derived compounds, indole 5carboxylic acid derivatives and 2,3-disubstituted indole 5-phenylacetic acid derivatives. It is significant that most of the PPARγ agonists exhibit substantial bioavailability following oral administration and have little or no toxicity associated with their use (See e.g., Saltiel and Olefsky, Diabetes 45:1661 (1996); Wang et al, Br. J. Pharmacol. 122:1405 (1997); and Oakes et al, Metabolism 46:935 (1997)). It will be appreciated that the present invention is not limited to above-identified PPARγ agonists and that other identified PPARγ agonists can also be used.

Compounds that can be used for practicing the present invention, and methods of making these compounds are disclosed in WO 91/07107; WO 92/02520; WO 94/01433; WO 89/08651; WO 96/33724; WO 97/31907; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; 5,260,445; 5,814,647; 5,902,726; 5,994,554; 6,294,580; 6,306,854; 6,498,174; 6,506,781; 6,541,492; 6,552,055; 6,579,893; 6,586,455, 6,660,716, 6,673,823; 6,680,387; 6,768,008; 6,787,551; 6,849,741; 6,878,749; 6,958,355; 6,960,604; 7,022,722 and U.S. Applications 20030130306, 20030134885, 20030109579, 20030109560, 20030088103, 20030087902, 20030096846, 20030092697, 20030087935, 20030082631, 2003007g288, 20030073862, 20030055265, 20030045553, 1 20020169192, 20020165282, 20020160997, 20020128260, 20020103188, 20020082292, 20030092736, 20030069275, 20020151569, and 20030064935.

The disclosures of these publications are incorporated herein by reference in their entireties, especially with respect to the PPARγ agonists disclosed therein, which may be employed in the methods described herein.

As agents having the aforementioned effects, the compounds of the following formulas are useful in treating individuals. Accordingly, in some embodiments of the present invention, the therapeutic agents comprise compounds of Formula I:

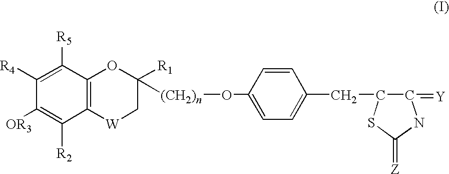

wherein R₁ and R₂ are the same or different, and each represents a hydrogen atom or a C₁-C₅ alkyl group; R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group; R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group; n is 1, 2, or 3; W represents the CH₂, CO, or CHOR₆ group (in which R₆ represents any one of the atoms or groups defined for R₃ and may be the same as or different, from R₃); and Y and Z are the same or different and each represents an oxygen atom or an imino (—NH) group; and pharmaceutically acceptable salts thereof.

In some embodiments of the present invention, the therapeutic agents comprise compounds of Formula II:

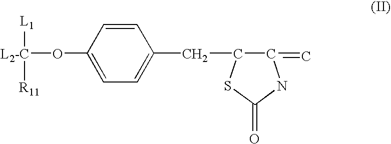

wherein R₁₁ is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in:

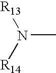

wherein R₁₃ and R₁₄ are the same or different and each is a lower alkyl (alternately, R₁₃ and R₁₄ are combined to each other either directly or as interrupted by a heteroatom comprising nitrogen, oxygen, and sulfur to form a 5- or 6-membered ring); and wherein L¹ and L² are the same or different and each is hydrogen or lower alkyl or L¹ and L² are combined to form an alkylene group; or a pharmaceutically acceptable salt thereof.

In some aspects of the present invention, the therapeutic agents comprise compounds of Formula III:

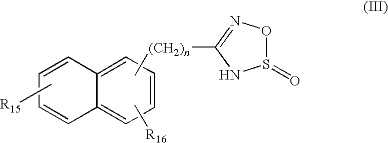

wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; n is 0 to 4; or a pharmaceutically acceptable salt thereof.

In some aspects of the present invention, the therapeutic agents comprise compounds of Formula IV:

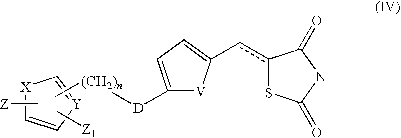

wherein the dotted line represents a bond or no bond; V is HCH—, —NCH—, —CH═N—, or S; D is CH₂, CHOH, CO, C═NOR₁₇, or CH═CH; X is S, SO, NR₁₈, —CH═N, or —N═CH; Y is CH or N; Z is hydrogen, (C₁-C₇)alkyl, (C₁-C₇)cycloalkyl, phenyl, naphthyl, pyridyl, furyl, thienyl, or phenyl mono- or di substituted with the same or different groups which are (C₁-C₃)alkyl, trifluoromethyl, (C₁-C₃)alkoxy, fluoro, chloro, or bromo; Z, is hydrogen or (C₁-C₃)alkyl; R₁₇ and R₁₈ are each independently hydrogen or methyl; and n is 1, 2, or 3; the pharmaceutically acceptable cationic salts thereof; and the pharmaceutically acceptable acid addition salts thereof when the compound contains a basic nitrogen.

In some embodiments of the present invention, the therapeutic agents comprise compounds of Formula V:

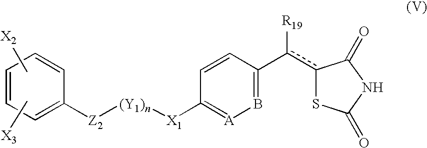

wherein the dotted line represents a bond or no bond; A and B are each independently CH or N. with the proviso that when A or B is N. the other is CH; X is S, SO, SO₂, CH₂, CHOH, or CO; n is 0 or 1; Y₁ is CHR₂₀ or R₂₁, with the proviso that when n is 1 and Y, is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂; R₁₉, R₂₀, R₂₁, and R₂₂ are each independently hydrogen or methyl; and X₂ and X₃ are each independently hydrogen, methyl, trifluoromethyl, phenyl, benzyl, hydroxy, methoxy, phenoxy, benzyloxy, bromno, chloro, or fluoro; a pharmaceutically acceptable cationic salt thereof; or a pharmaceutically acceptable acid addition salt thereof when A or B is N.

In some embodiments of the present invention, the therapeutic agents comprise compounds of Formula VI:

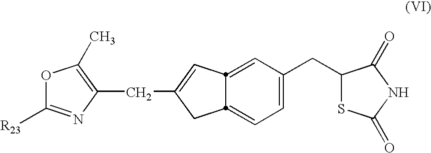

or a pharmaceutically acceptable salt thereof, wherein R₂₃ is alkyl of 1 to 6 carbon atoms, cycloalkyl of 3 to 7 carbon atoms, phenyl or mono- or all-substituted phenyl wherein said substituents are independently alkyl of 1 to 6 carbon atoms, alkoxy of 1 to 3 carbon atoms, halogen, or trifluoromethyl.

In some embodiments of the present invention, the therapeutic agents comprise compounds of Formula VII:

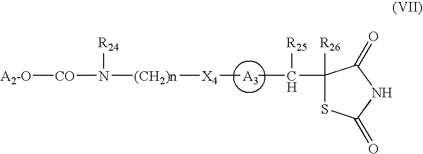

or a tautomeric form thereof and/or a pharmaceutically acceptable salt thereof, and/or a pharmaceutically acceptable solvate thereof, wherein: A₂ represents an alkyl group, a substituted or unsubstituted aryl group, or an aralkyl group wherein the alkylene or the aryl moiety may be substituted or unsubstituted; A³ represents a benzene ring having in total up to 3 optional substituents; R₂₄ represents a hydrogen atom, an alkyl group, an acyl group, an aralkyl group wherein the alkcyl or the aryl moiety may be substituted or unsubstituted, or a substituted or unsubstituted aryl group; or A₂ together with R₂₄ represents substituted or unsubstituted C₂₋₃ polymethylene group, optional substituents for the polymethylene group being selected from alkyl or aryl or adjacent substituents together with the methylene carbon atoms to which they are attached form a substituted or unsubstituted phenylene group; R₂₅ and R₂₆ each represent hydrogen, or R₂₅ and R₂₆ together represent a bond; X₄ represents O or S; and n represents an integer in the range from 2 to 6.

In some embodiments of the present invention, the therapeutic agents comprise compounds of Formula VIII:

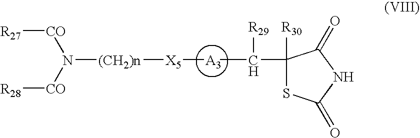

or a tautomeric form thereof and/or a pharmaceutically acceptable salt thereof, and/or a pharmaceutically acceptable solvate thereof, wherein: R₂₇ and R₂₈ each independently represent an alkyl group, a substituted or unsubstituted aryl group, or an aralkyl group being substituted or unsubstituted in the aryl or alkyl moiety; or R₂₇ together with R₂₈ represents a linking group, the linking group consisting or an optionally substituted methylene group or an O or S atom, optional substituents for the methylene groups including alkyl, aryl, or aralkyl, or substituents of adjacent methylene groups together with the carbon atoms to which they are attached form a substituted or unsubstituted phenylene group; R₂₉ and R₃₀ each represent hydrogen, or R₂₉ and R₃₀ together represent a bond; A₄ represents a benzene ring having in total up to 3 optional substituents; X₅ represents O or S; and n represents an integer in the range of 2 to 6.

In some embodiments of the present invention, the therapeutic agents comprise compounds of Formula IX:

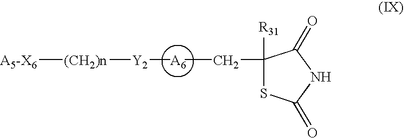

or a tautomeric form thereof and/or a pharmaceutically acceptable salt thereof, and/or a pharmaceutically acceptable solvate thereof, wherein: A₅ represents a substituted or unsubstituted aromatic heterocyclyl group; A₆ represents a benzene ring having in total up to 5 substituents; X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group; Y₂ represents O or S; R₃, represents an alkyl, aralkyl, or aryl group; and n represents an integer in the range from 2 to 6. Aromatic heterocyclyl groups include substituted or unsubstituted, single or fused ring aromatic heterocyclyl groups comprising up to 4 hetero atoms in each ring selected from oxygen, sulfur, or nitrogen. Aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulfur, or nitrogen. Values for A₅ when it represents a 5-membered aromatic heterocyclyl group include thiazolyl and oxazoyl, especially oxazoyl. Values for A₆ when it represents a 6 membered aromatic heterocyclyl group include pyridyl or pyrimidinyl. R₃, represents an alkyl group, in particular a C-6 allyl group (e.g., a methyl group).

A⁵ can represent a moiety of formula (a), (b), or (c), under Formula IX:

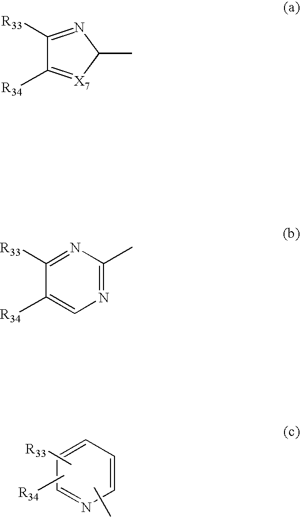

wherein, R₃₃ and R₃₄ each independently represents a hydrogen atom, an alkyl group, or a substituted or unsubstituted aryl group or when R₃₃ and R₃₄ are each attached to adjacent carbon atoms, then R₃₃ and R₃₄ together with the carbon atoms to which they are attached forth a benzene ring wherein each carbon atom represented by R₃₃ and R₃₄ together may be substituted or unsubstituted; and in the moiety of Formula (a), X₇ represents oxygen or sulphur.

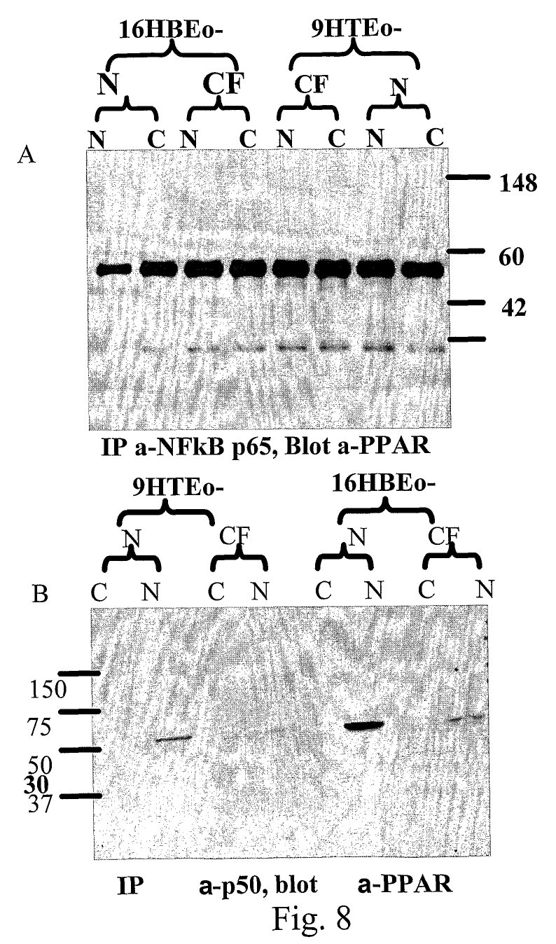

In one embodiment of the present invention, R₃₃ and R₃₄ together present a moiety of Formula (d) in FIG. 8, under Formula IX:

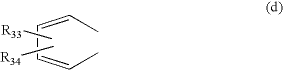

wherein R₃₅ and R₃₆ each independently represent hydrogen, halogen, substituted or unsubstituted alkyl, or alkoxy.

In some embodiments of the present invention, the therapeutic agents comprise compounds of Formula X:

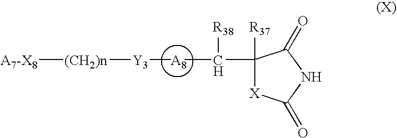

or a tautomeric form thereof and/or a pharmaceutically acceptable salt thereof, and/or a pharmaceutically acceptable solvate thereof, wherein: A₇ represents a substituted or unsubstituted aryl group; A₈ represents a benzene ring having in total up to 5 substituents; X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group; Y₃ represents O or S; R₃₇ represents hydrogen; R₃₈ represents hydrogen or an alkyl, aralkyl, or aryl group or R₃₇ together with R₃₈ represents a bond; and n represents an integer in the range from 2 to 6.

In some embodiments of the present invention, the therapeutic agents comprise compounds of Formula XI:

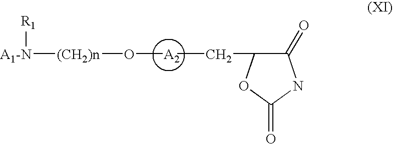

or a tautomeric form thereof and/or a pharmaceutically acceptable salt thereof, and/or a pharmaceutically acceptable solvate thereof, wherein: A¹ represents a substituted or unsubstituted aromatic heterocyclyl group; R₁ represents a hydrogen atom, an alkyl group, an acyl group, an aralkyl group, wherein the aryl moiety may be substituted or unsubstituted, or a substituted or unsubstituted aryl group; A₂ represents a benzene ring having in total up to 5 substituents; and n represents an integer in the range of from to 6. Suitable aromatic heterocyclyl groups include substituted or unsubstituted, single or fused ring aromatic heterocyclyl groups comprising up to 4 hetero atoms in each ring selected from oxygen, sulfur, or nitrogen. Favored aromatic heterocyclyl groups include substituted or unsubstituted single ring aromatic heterocyclyl groups having 4 to 7 ring atoms, preferably 5 or 6 ring atoms. In particular, the aromatic heterocyclyl group comprises 1, 2, or 3 heteroatoms, especially 1 or 2, selected from oxygen, sulfur, or nitrogen. Values for A₁ when it represents a 5-membered aromatic heterocyclyl group can include thiazolyl and oxazolyl, especially oxazoyl. Values for A₁ when it represents a 6-membered aromatic heterocyclyl group can include pyridyl or pyrimidinyl.

In some embodiments of the present invention, the therapeutic agent comprises a compound of Formulas XII and XIII:

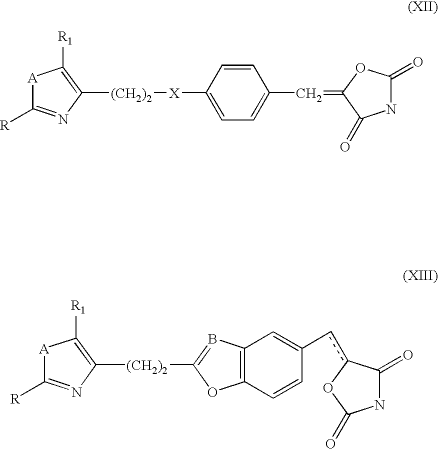

or pharmaceutically acceptable salts thereof wherein the dotted line represents a bond or no bond; R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl); R₁ is an alkyl of one to three carbon atoms; X is O or C═O; A is O or S; and B is N or CH.

Some embodiments of the present invention include the use of the compounds of Formulas I through XIII are referred to as thiazolidine derivatives. Where appropriate, the specific names of thiazolidine derivatives may be used including: troglitazone, ciglitazone, pioglitazone, and rosiglitazone.

In certain embodiments, the therapeutic agent comprises an activator of PPARγ as described in U.S. Pat. No. 5,994,554, e.g., having a structure selected from the group consisting of formulas (XIV)-(XXVI):

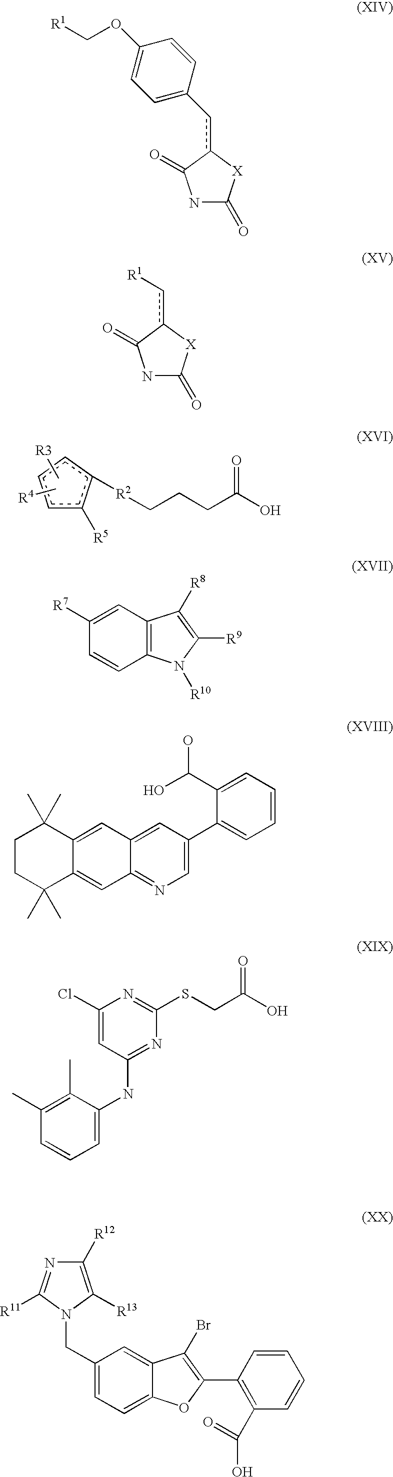

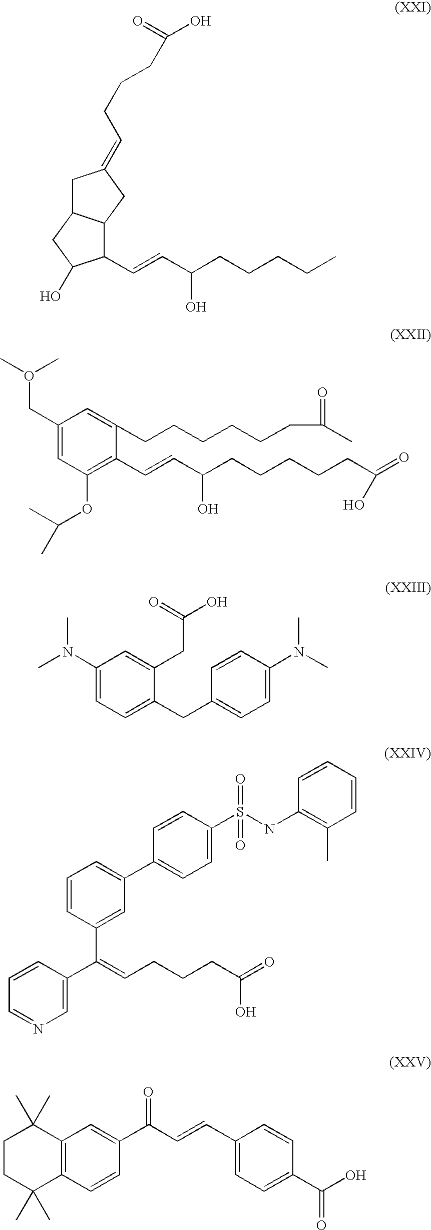

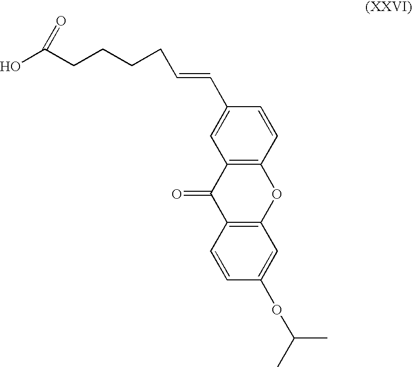

wherein: R¹ is selected from the group consisting of hydrogen, C₁₋₈ alkyl, aminoC₁₋₈, alkyl, C₁₋₈alkylamino C₁₋₈ alkyl, heteroarylamino C₁₋₆ alkyl, (heteroaryl)(C₁₋₈alkyl)aminoC₁₋₆ alkyl, (C₁₋₈ cycloalkyl) C₁₋₈ alkyl, C₁₋₈ alkylheteroaryl C₁₋₈ alkyl, 9- or 10-membered heterobicycle, which is partially aromatic or substituted 9- or 10-membered heterobicycle, which is partially aromatic; X is selected from the group consisting of S, NH, or O; R² is selected from the group consisting of hydrogen, C₁₋₈allyl or C₁₋₈alkenyl; R³ and R⁴ are independently selected from the group consisting of hydrogen, hydroxy, oxo C₁₋₈alkyl, C₁₋₈alkoxy or amino; R⁵ is selected from the group consisting of hydrogen, C₁₋₈alkyl, C₁₋₈alkenyl, (carbonyl)alkenyl, (hydroxy)alkenyl, phenyl, C₁₋₈alkylR⁶, (hydroxy) C₁₋₈alkylR⁶, C₁₋₈alkyl C₁₋₈cycloallylR⁶, (hydroxy) C₁-C₁₋₈cycloallylR⁶ or C₁₋₈cycloallylthioR⁶; R⁶ is selected from the group consisting of phenyl or phenyl substituted with hydroxy, C₁₋₈alkyl or C₁₋₈alkoxy substituents; R⁷ is selected from the group consisting of hydrogen, hydroxy, carboxy or carboxy C₁₋₈alkyl; R¹ is selected from the group consisting of hydrogen, C₁₋₈alkyl, phenyl, phenyl C₁₋₈alkyl, phenyl mono- or all-substituted with halo, hydroxy, and/or C₁₋₈alkoxy (e.g., methoxy) substituents or phenyl C₁₋₈alkyl wherein the phenyl is mono- or disubstituted with halo, hydroxy, and/or C₁₋₈alkoxy (e.g., methoxy) substituents; R⁹ is selected from the group consisting of hydrogen, C₁₋₈alkyl, carboxy C₁₋₈alkenyl mono- or disubstituted with hydroxy, and/or C₁₋₈alkoxy (e.g., methoxy), phenyl or phenyl mono- or disubstituted with halo, hydroxy, and/or C₁₋₈alkoxy (e.g., methoxy) R¹⁰ is hydrogen or C₁₋₈alkyl, R¹¹ is selected from the group consisting of hydrogen, C₁₋₈alkyl or cycloC₁₋₈alkyl C₁₋₈alkyl; R¹² is selected from the group consisting of hydrogen, halo or fluorinated C₁₋₈alkyl; R¹³ is selected from the group consisting of hydrogen, C₁₋₈alkoxycarbonyl or C₁₋₈alkoxycarbonyl C₁₋₈alkylaminocarbonyl; a dashed line ( - - - ) is none or one double bond between two of the carbon atoms; fluorinated alkyl can be an alkyl wherein one or more of the hydrogen atoms is replaced by a fluorine atom; heteroaryl can be 5, 6 or 7 membered aromatic ring optionally interrupted by 1, 2, 3 or 4 N, S, or O heteroatoms, with the proviso that any two O or S atoms are not bonded to each other; substituted heteroaryl can be a 9- or 10-membered heterobicycle mono-, di-, or trisubstituted independently with hydroxy, oxo, C₁₋₁₆ alkyl, C₁₋₆ alkoxy or 9- or 10-membered heterobicycle, which is partially aromatic in more detail is a heterobicycle interrupted by 1, 2, 3, or 4 N heteroatoms; substituted 9- or 10-membered heterobicycle, which is partially aromatic in more detail is a 9- or 10-membered heterobicycle mono-, di-, tri- or tetrasubstituted independently with hydroxy, oxo, C₁₋₈ alkyl, C₁₋₈ alkoxy, phenyl, phenyl C₁₋₈ alkyl; or a pharmaceutically acceptable acid-addition or base-addition salt thereof.

In yet other embodiments, the therapeutic agent comprises a compound as disclosed in U.S. Pat. No. 6,306,854, e.g., a compound having a structure of Formula (XXVII):

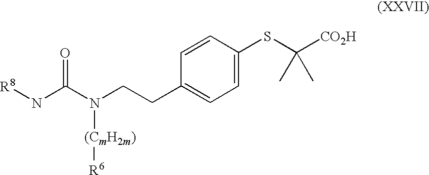

and esters, salts, and physiologically functional derivatives thereof; wherein m is from 0 to 20, R⁶ is selected from the group consisting of hydrogen and

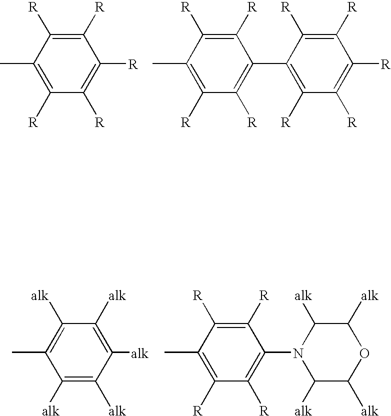

and R⁸ is selected frown the group consisting of:

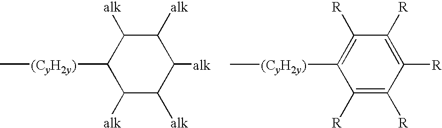

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and which can contain hetero atoms such as nitrogen, oxygen, or sulfur and which can contain functional groups such as ketone or ester, cycloalkyl containing 3 to 7 carbon atoms, or two R groups bonded to adjacent carbon atoms can, together with the carbon atoms to which they are bonded, form an aliphatic or aromatic ring or multi ring system, and where each depicted ring has no more than 3 alk groups or R groups that are not hydrogen.

In yet other embodiments of the present invention a therapeutic agent is a compound such as disclosed in U.S. Pat. No. 6,294,580 and/or Liu et al., Biorg. Med. Chem. Lett. 11 (2001) 3111-3113, e.g., having a structure within Formula XXVIII:

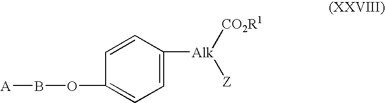

wherein A is selected from the group consisting of: (i) phenyl, wherein said phenyl is optionally substituted by one or more of the following groups; halogen atoms, C₁₋₆alkyl, C₁₋₃ alkoxy, C₁₋₃ fluoroalkoxy, nitrite, or —NR⁷R⁸ where R⁷ and R⁸ are independently hydrogen or C₁₋₃ aRlyl; (ii) a 5- or 6-membered heterocyclic group containing at least one heteroatom selected from oxygen, nitrogen and sulfur; and (iii) a fused bicyclic ring

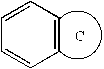

wherein ring C represents a heterocyclic group as defined in point (ii) above, which bicyclic ring is attached to group B via a ring atom of ring C; B is selected from the group consisting of: (iv) C₁₋₆ alkylene; (v) -M C₁₋₆ alkylene or C₁₋₆ alkyleneM C₁₋₆ alkylene, wherein M is O, S, or —NR² wherein R² represents hydrogen or C₁₋₃ alkyl; (vi) a 5- or 6-membered heterocyclic group containing at least one nitrogen heteroatom and optionally at least one further heteroaton selected from oxygen, nitrogen and sulfur and optionally substituted by C₁₋₃ alkyl; and (vii) Het-C₁₋₆ allylene, wherein Het represents a heterocyclic group as defined in point (vi) above; Alk represents C₁₋₃ alkylene; Het represents hydrogen or C₁₋₃ alkyl; Z is selected from the group consisting of: (viii) nitrogen-containing heterocyclyl or heteroaryl, e.g., N-pyrrolyl, N-piperidinyl, N-piperazinyl, N-morpholinyl, or N-imidazolyl, optionally substituted with 1-4 C₁₋₆ alkyl or halogen substituents; (ix) —(C₁₋₃ alkylene) phenyl, which phenyl is optionally substituted by one or more halogen atoms; and (x) —NR³R⁴, wherein R³ represents hydrogen or C₁₋₃ alkyl, and R⁴ represents C₁₋₆ alkyl, aryl or heteroaryl (e.g., phenyl, pyridinyl, pyrazinyl, pyrimidinyl, pyrrolyl, piperidinyl, piperazinyl, morpholinyl, imidazolyl), optionally substituted by 1-4 C₁₋₆ allyl, halogen, C₁₋₆ alkoxyl, hydroxyl, nitro, cyano, or amino substituents, or —Y—(C═O)-T-R⁵, —Y—SO₂—R⁵, or —Y—(CH(OH))-T-R⁵, wherein: (a) Y represents a bond, C₁₋₆ alkylene, C₂₋₆ alkenylene, C₄₋₆ cycloalkylene or cycloallkenylene, a heterocyclic group as defined in point (vi) above, or phenyl optionally substituted by one or more C₁₋₃ alkyl groups and/or one or more halogen atoms; (b) T represents a bond, C₁₋₃ alkyleneoxy, —O— or —N(R⁶)—, wherein R⁵ represents hydrogen or C₁₋₃ allyl; (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₁₋₃ alkyleneNR⁹R′″ (where each R⁹ and R¹⁰ is independently hydrogen, C₁₋₃ alkyl, —SO₂C₁₋₃ alkyl, or —CO₂C₁₋₃ allyl, —SO₂ NHC₁₋₃ alkyl), C₁₋₃ alkyleneCO₂H, C₁₋₃allyleneCO₂C₁₋₃ alkyl, or —OCH₂C(O)NH₂), a 5- or 6 membered heterocyclic group as defined in point (ii) above, a bicylic fused ring

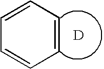

wherein ring D represents a 5- or 6-membered heterocyclic group containing at least one heteroatom selected from oxygen, nitrogen and sulfur and optionally substituted by (═O), which bicyclic ring is attached to T via a ring atom of ring D: or —C₁₋₆ alkyleneMR¹¹M is O, S, or —NR¹² wherein R¹¹ and R¹² are independently hydrogen or C₁₋₃ alkyl, or a tautomeric form thereof, and/or a pharmaceutically acceptable salt or solvate thereof.

One specific group of compounds are those of Formula XI, wherein the dotted line represents no bond, R¹ is methyl, X is O and A is O. Examples of compounds in this group are those compounds where R is phenyl, 2-naphthyl and 3,5 bis(trifluoronethyl)phenyl. Another specific group of compounds are those of Formula XIII, wherein the dotted line represents no bond, R¹ is methyl and A is O. Particularly preferred compounds within this group are compounds where B is CH and R is phenol, p-tolyl, m-tolyl, cyclohexyl, and 2-naphthyl. In alternative embodiments of the present invention, the B is N and R is phenyl.

In still further embodiments, the present invention provides methods for the use of a pharmaceutical composition suitable for administering an effective amount of at least one composition comprising a PPARγ agonist, such as those disclosed herein, in unit dosage form to treat cystic fibrosis related disorders and/or inflammation associated with NF-κB activation. In alternative embodiments, the composition further comprises a pharmaceutically acceptable carrier.

Specific examples of compounds of the present invention are given in the following list: (+)-5[[4-[(3,4-dihydro-6-hydroxy-2,5,7,8-tetramethyl-2H-1-benzopyran-2-yl)methoxy]phenyl]methyl]-2,4thiazolidinedione; (Troglitazone); 5-[4-[2-(5-ethylpyridin-2-yl)ethoxyl]benzyl]thiazolidine-2,4-dione; (pioglitazone); 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chlorophyll)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; (englitazone); 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione (rosiglitazone); and 5-[4-[2-(N-(benzoxazol-2-yl)-N-methylamino]ethoxy]benzyl]oxazolidine-2,4-dione.

In yet other embodiments of the present invention, the therapeutic agents comprise compounds having the structure shown in Formula XXIX:

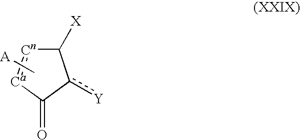

wherein: A is selected from hydrogen or a leaving group at the α- or β-position of the ring, or A is absent when there is a double bond between the C^{a} and Cⁿ of the ring; X is an alkyl, substituted alkyl, alkenyl, substituted alkenyl, alkynyl, or substituted alkynyl group having in the range of 2 up to 15 carbon atoms; and Y is an alkyl, substituted alkyl, alkenyl, substituted alkenyl, alkynyl, or substituted alkynyl group having in the range of 2 up to 15 carbon atoms. As used herein, the term “leaving group” refers to functional groups which can readily be removed from the precursor compound, for example, by nucleophilic displacement, under E2 elimination conditions, and the like. Examples include, but are limited to, hydroxy groups, alkoxy groups, tosylates, brosylates, halogens, and the like.

The therapeutic agents of the present invention (e.g., the compounds in Formulas I-XXIX and the others described above) are capable of further forming both pharmaceutically acceptable acid addition and/or base salts. All of these forms are within the scope of the present invention and can be administered to the subject to treat cystic fibrosis related disorders and inflammation associated with NF-κB activation. Pharmaceutically acceptable acid addition salts of the present invention include, but are not limited to, salts derived from nontoxic inorganic acids such as hydrochloric, nitric, phosphohoric, sulfuric, hydrobromic, hydriodic, hydrofluoric, phosphorous, and the like, as well as the salts derived forth nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bissulfite, nitrate, phosphate, monoLydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malcate, maudelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like, as well as gluconate, galacturonate, and n-methyl glucamie.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner or as described above. The free base forms differ from their respective salt forms somewhat in certain physical properties such as solubility in polar solvents, but are otherwise equivalent to their respective free base for purposes of the present invention.

Pharmaceutically acceptable base addition salts are formed with metals or amides, such as alkali and alkaline earth metals or organic amines. Examples of metals used as cations include, but are not limited to, sodium, potassium, magnesium, calcium, and the like. Examples of suitable amines include, but are not limited to, N2 N′-dibelizylethylenediamine, chloroprocaine, choline, diethanolamine, dicyclohexylamine, ethylenediamine, N-methylglucamine, and procaine

The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional marnuer or as described above. The free acid forms differ from their respective salt forms somewhat in certain physical properties such as solubility in polar solvents, but otherwise the salts are equivalent to their respective free acid for purposes of the present invention.

Certain of the compounds of the present invention can exist in unsolvated forms as well as solvated forms, including, but not limited to, hydrated forms In general, the solvated forms, including hydrated forms, are equivalent to unsolvated forms and are intended to be encompassed within the scope of the present invention. Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereospecific synthesis tecdiques are employed or optically active compounds are employed as starting materials in the preparation of the compounds, individual isomers may be prepared directly. However, if a mixture of isomers is prepared, the individual isomers may be obtained by conventional resolution techniques, or the mixture may be used as is, with resolution.

Furthermore, the thiazolidene or oxazolidene part of the compounds of Formulas I through XIII can exist in the form of tautomeric isomers, and are intended to be a part of the present invention.

For preparing pharmaceutical compositions from the compounds of the present invention, pharmaceutically acceptable carriers can be in any suitable form (e.g., solids, liquids, gels, etc.). Solid form preparations include, but are not limited to, powders, tablets, pills, capsules, cachets, suppositories, and dispersible granules. A solid carrier can be one or more substances which may also act as diluents, flavoring agents, binders, preservatives, tablet disintegrating agents, or an encapsulating material. The present invention contemplates a variety of techniques for administration of the therapeutic compositions. Suitable routes include, but are not limited to, oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or iltraocular injections, among others. Indeed, it is not intended that the present invention be limited to any particular administration route.

For injections, the agents of the present invention may be formulated in aqueous solutions, preferably in physiologically compatible buffers such as Hank's solution, Ringer's solution, or physiological saline buffer.

For such transmucosal administration, penetrants appropriate to the barrier to be permeated are used in the formulation. Such penetrants are generally known in the art.

In powders, the carrier is a finely divided solid which is in a mixture with the finely dived active component. In tablets, the active component is mixed with the carrier having the necessary binding properties in suitable proportions, which has been shaped into the size and shape desired.

The powders and tablets preferably contain from five or ten to about seventy percent of the active compounds. Suitable carriers include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter and the like, among other embodiments (e.g., solid, gel, and liquid forms). The term “preparation” is intended to also encompass the formation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, in some embodiments of the present invention, a low melting wax, such as a mixture of fatty acid glycerides or cocoa butter; is first melted and the active compound is dispersed homogeneously therein, as by stirring.

The molten homogenous mixture is then poured into convenient sized molds, allowed to cool, and thereby to solidify in a form suitable for administration.

Liquid form preparations include, but are not limited to, solutions, suspensions, and emulsions (e.g., water or water propylene glycol solutions). For parenteral injection, in some embodiments of the present invention, liquid preparations are formulated in solution in aqueous polyethylene glycol solution. Aqueous solutions suitable for oral use can be prepared by dissolving the active component in water and adding suitable colorants, flavors, and stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into limit doses containing appropriate quantities of the active component. The unit dosage form can be a packaged preparation, the package containing discrete quantities of preparation, such as paclceted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 100 mg, preferably ranging from 0.5 mg to 100 mg according to the particular application and the potency of the active component. The composition can, if desired, also contain other compatible therapeutic agents.

General procedures for preparing pharmaceutical compositions are described in Remington's Pharmaceutical Sciences, E. W. Martin ea., Mack Publishing Co., PA (1990).

The invention herein involves a method of treatment of cystic fibrosis related disorders using an aerosol formulation which comprises (a) one or more PPARγ agonists; and (b) a suitable fluid carrier. See, for example, U.S. Pat. Nos. 6,613,307; 6,610,272; and, 6,596,261.

The aerosol formulation of the invention can be prepared by combining (a) PPARγ agonists in an amount sufficient to provide a plurality of therapeutically effective doses; (b) the propellant, in an amount sufficient to propel a plurality of doses from an aerosol canister; (c) optionally, the water addition in an amount effective to further stabilize each of the formulations; and (d) any further optional components, such as, for example, ethanol as a cosolvent; and dispersing the components. The components can be dispersed using a conventional mixer or homogenizer, by shaking, or by ultrasonic energy as well as by the use of a bead mill or a microfluidizer. Bulk formulations can be transferred to smaller individual aerosol vials by using valve to valve transfer methods, pressure filling or by using conventional cold-fill methods. See, for example, U.S. Pat. Nos. 6,613,307; 6,610,272; and, 6,596,261.

It is not required that a component used in a suspension aerosol formulation be soluble in the fluid carrier, such as a propellant. Components that are not sufficiently soluble can be coated or congealed with polymeric, dissolution controlling agents in an appropriate amount and the coated particles can then be incorporated in a formulation as described above. Polymeric dissolution controlling agents suitable for use in this invention include, but not limited to polylactide glycolide co-polymer, acrylic esters, polyamidoamines, substituted or unsubstituted cellulose derivatives, and other naturally derived carbohydrate and polysaccharide products such as zein and chitosan. See, for example, U.S. Pat. Nos. 6,613,307; 6,610,272; and, 6,596,261.

Therapeutic agents are commonly administered to the lung in the form of an aerosol of particles of respirable size (less than about 10 μm in diameter). The aerosol PPARγ agonist formulation can be presented as a liquid or a dry powder. In order to assure proper particle size in a liquid aerosol, particles can be prepared in a size suitable for respiration and then incorporated into a colloidial dispersion either containing a propellant as a metered dose inhaler (MDI) or air, such as in the case of a dry powder inhaler (DPI). Alternatively, the PPARγ agonist formulations can be prepared in solution form in order to avoid the concern for proper particle size in the formulation. Solution formulations of PPARγ agonists must nevertheless be dispensed in a manner that produces particles or droplets of respirable size. For MDI application, once prepared, an aerosol formulation is filled into an aerosol canister equipped with a metered dose valve. See, for example, U.S. Pat. Nos. 6,613,307; 6,610,272; and, 6,596,261. For purposes of the methods of administration, the PPARγ agonists can also be micronized whereby a therapeutically effective amount or fraction of the PPARγ agonist is particulate. Typically, the particles have a diameter of less than about 10 microns, and preferably less than about 5 microns, in order that the particles can be inhaled into the respiratory tract and/or lungs.

A number of medicinal aerosol formulations using propellant systems are disclosed in, for example, U.S. Pat. No. 6,613,307 and the references cited therein (such as, for example, EP 0372777, WO91/04011, WO91/11173. WO91/11495, WO91/14422, WO92/00107, WO93/08447, WO93/08446. WO93/11743, WO93/11744 and WO93/11745) all of which are incorporated by reference herein in their entirety. Many such propellants are known in the art and are suitable for use in the invention herein. The propellants for use in the invention may be any fluorocarbon, hydrogen-containing fluorocarbon or hydrogen-containing chlorofluorocarbon propellant or mixtures thereof having a sufficient vapour pressure to render them effective as propellants. Suitable propellants include, for example, chlorofluorocarbons. The propellant may additionally contain a volatile adjuvant such as a saturated hydrocarbon for example propane, n-butane, isobutane, pentane and isopentane or a dialkyl ether for example dimethyl ether.

Where a surfactant is employed in the aerosol, it is selected from those which are physiologically acceptable upon administration by inhalation such as oleic acid, sorbitan trioleate (Span R 85), sorbitan mono-oleate, sorbitan monolaurate, polyoxyethylene, sorbitan monolaurate, polyoxyethylene, sorbitan monooleate, natural lecithin, fluorinated and perfluorinated surfactants including fluorinated lecithins, fluorinated phosphatidylcholines, oleyl polyoxyethylene ether, stearyl polyoxyethylene ether, lauryl polyoxyethylene ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil and sunflower seed oil. See, for example, U.S. Pat. No. 6,613,307.

Also provided herein for use in the methods are aerosol formulations, which contain PPARγ agonists and additionally one or more therapeutic agents. The additional therapeutic agents may be selected from any other suitable drug useful in inhalation therapy and which may be presented in a form, which is substantially completely insoluble in the selected propellant. Where appropriate, the PPARγ agonists may be used in the form of salts, esters or as solvates to optimize the activity and/or stability of the PPARγ agonists and/or to minimize the solubility of the PPARγ agonists in the propellant. See, for example, U.S. Pat. No. 6,613,307.

The assessment of the clinical features and the design of an appropriate therapeutic regimen for the individual patient is ultimately the responsibility of the prescribing physician. It is contemplated that, as part of their patient evaluations, the attending physicians know how to and when to terminate, interrupt, or adjust administration due to toxicity, or to organ dysfunctions. Conversely, the attending physicians also know to adjust treatment to higher levels, in circumstances where the clinical response is inadequate, while precluding toxicity. The magnitude of an administrated dose in the management of the disorder of interest will vary with the severity of the condition to be treated, the patient's individual physiology, biochemistry, etc., and to the route of administration. The severity of the condition, may, for example, be evaluated, in part, by standard prognostic evaluation methods.

Further, the dose and dose frequency will also vary according to the age, body weight, sex and response of the individual patient.

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); EM (micromolar); mol (moles); mmol (millimoles); μmolcromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); Sigma (Sigma Chemical Co., St. Louis, Mo.), parts per million (ppm).

### EXAMPLE

### Inflammatory Stimuli Alter the Interaction of PPARγ with Binding Partners in Airway

Epithelial Cells Comparison of Cystic Fibrosis (CF) Cells v. Non-Cystic Fibrosis Cells and Animals

The CF airway epithelial cell responds to inflammatory stimuli with increased production of proinflammatory cytokine IL-8, as well as IL-6 and GM-CSF compared to normal controls, as a result of increased activation of NF-κB in the CF cells. In order to investigate mechanisms by which NF-κB could be activated in excess in CF, and potential therapeutic interventions to prevent this excessive activation, we assessed PPARγ in airway epithelium. In CF, PPARγ function is reduced. This may contribute to the excess NF-κB activation because PPARγ interacts with NF-κB to prevent its function as a transcription factor. Under conditions of inflammatory stimulation, such as PAO1 exposure or TNFα/IL-1β treatment, the interaction between PPARγ and NF-κB is reduced, but this reduction is abrogated by administration of PPARγ agonists. In vivo, administration of PPARγ agonists results in reduced airway inflammation in response to acute administration of P. aeruginosa in CF, but not wild type, mice. Taken together these data indicate that PPARγ influences the inflammatory response at the level of NF-κB in airway epithelial cells, and it may be a therapeutic target in CF.

In cystic fibrosis (CF), inflammation is an independent contributor to the decline in pulmonary function and a valid therapeutic target. In vivo studies in infants and children, nearly all studies in CF mice, and many studies in CF airway epithelial cell cultures and cell lines show that the inflammatory response, either to TNFα and IL1β or to P. aeruginosa, occur in excess in CF. The cytokines that are most consistently in excess in CF (e.g., IL-8 or murine equivalents, IL-6, GM-CSF) require activation of NF-κB for upregulation, and several laboratories have shown increased activation of NF-κB in CF airway epithelial cell lines. Failure to appropriately modulate activation of NF-κB could account for the excess inflammatory response in CF, and control of activation of NF-κB could be therapeutic.

The expression and role of PPARγ in airway epithelial cells has not been elucidated. Because inflammation is an important part of the CF lung disease, and because expression of PPARγ has been shown to be reduced in organs known to express CFTR in CF mice, we tested the role of PPARγ in airway epithelial cells of CF and non-CF phenotype with respect to the inflammatory response, and in CF and non-CF mice challenged with the CF pathogen, Pseudomonas aeruginosa. We found that PPARγ is expressed in human airway epithelial cells in culture and in vivo in mouse airway epithelium. The DNA binding properties of PPARγ are activated in response to challenge with P. aeruginosa. However, PPARγ also interacts with other transcription factors, including NF-κB, and this interaction is reduced by inflammatory stimuli such as P. aeruginosa or TNF-α/IL-1β. Activation of PPARγ with its agonists can restore interaction with other transcription factors, including NF-κB, and also inhibits release of inflammatory mediators and proteins by airway epithelial cells. Here we test the hypothesis that the activation of NF-κB observed in CF epithelial cells can be accounted for in part by reduced binding to PPARγ, and that activation of PPARγ in airway epithelium can prevent excess activation of NF-κB. We also test whether, in an animal model of acute pseudomonas infection, PPARγ agonists will reduce the inflammatory response. Our results show that activation of PPAR-γ can be of therapeutic use in modulating the excess inflammatory response associated with CF.

### Methods

Cell lines: 16HBE-S and 16HBE-AS cells were grown in EMEM supplemented with 10% fetal bovine serum, 2 mM L-glutamine, 100 units/100 μg per ml of penicillin-streptomycin and 400 μg/ml G-418 as previously described.

### Well-Differentiated Human Airway Epithelial Cells Grown at the Air-Liquid Interface:

Human tracheal epithelial cells (HTE) recovered from necropsy specimens were grown in an air-liquid interface (ALI) on collagen-coated, semipermeable membranes (either 7×10⁶ cells/4.5 cm filter or 1×10⁶ cells/1 cm² filter, transwell-clear polyester membrane, Costar, Corning, N.Y.) and allowed to differentiate in serum-containing media for three or four weeks.

At three or four weeks, on day 0, cells are switched to submerged culture (liquid-liquid interface, LLI) and treated with either DMSO 1:1000 (vehicle control, normal cells, Sigma, St. Louis, Mo.), or 20 μM CFTRᵢₙₕ-172 (kindly provided by Alan Verkman) prepared in DMSO, and diluted from a 1:1000 stock. Drugs are added to both the basolateral (1 or 2 ml volume, according to filter size) and the apical side (0.35 or 1.5 ml) and media replenished every 24H. Cells grown in this way with I172 have been shown to have continuous inhibition of CFTR activity >90% but no decrease in cell viability or change in cell morphology by electron microscopy. These cells do not display increased amiloride-sensitive sodium conductance. Moreover, we have shown that cells grown in this way with I172 have increased basal and stimulated secretion of IL-8, increased activated RhoA, and decreased Smad3 expression on day 3, and that these changes are not the direct result of I172 on cytokine synthesis per se, since they do not occur in CF cells. At day 3, cells were committed to inflammatory stimulation with TNF IL-1 or PAO1 as indicated below. Cells committed to PAO1 stimulation were switched to serum-free media 24H prior PAO1 stimulation, kept in serum-free media until the end of the experiment, and media replenished every 12H during that time. Serum-free media contained 1:1 DMEM-Ham's F-12, pH. 7.2, L-glutamine 2.5 mM, Penicillin/Streptomycin 100 units/100 μg per ml, gentamicin 50 μg/ml, amphotericin B 1.25 μg/ml from Gibco, Invitrogen Corporation, Carlsbad, Calif.; fluconazole 2 μg/ml (DIFLUCON®, Pfizer); transferrin 5 μg/ml, hydrocortisone 5 μM, insulin 5 μg/ml, endothelial cell growth supplement 20 μg/mil, and bovine serum albumin 1 mg/ml from Sigma, St. Louis, Mo. Serum-containing media had the same antibiotics present as the serum-free media. Antibiotics were not present during PAO1 stimulation.

In order to observe the localization of the subunits of NF-κB by immunohistochemistry in well-differentiated airway epithelial cells, the cultures were dissociated and single cells transferred to chamber slides and allowed to adhere. This transfer was performed because cells in the well-differentiated model that had been maintained for more than two weeks displayed heaping of cells so that the nuclei could not be located in a single focal plane, making assessment of nuclear translocation of the proteins difficult. Cells were then treated either with vehicle, or TNF/IL1 for 15 min, fixed, permeabilized, and stained with antibody to the p50 and p65 subunits of NF-κB, and DAPI to locate nuclei.

Zymography: Supernatants of cells were centrifuged for 10 minutes at 14,000 rpm. Supernatants were then concentrated with an Ultra-4 Filter (Amicon) to 50-70 ul. Protein assays were done with Protein Assay Reagent (Bio-Rad). Cell supernatants were mixed with 2× nonreducing SDS sample buffer. Standard SDS-PAGE gels were prepared containing 1 mg/mil gelatin. To allow MMPs to renature, gels were washed twice in 2.5% TX-100 in sterile water. Gels were incubated in activation buffer (10 mM Tris-HCl, pH 7.5, 1.25% TX-100, 5 mM CaCl₂, 1 uM ZnCl₂) overnight at 37° C. Staining with 0.25% Coomassie brilliant blue R-250 diluted in 40% methanol and 10% acetic acid required 1-2 hours. Gels were destained in 40% methanol and 10% acetic acid until clear zones of protease activity are visible in a blue background.

Reporter Gene Assays: Cells were seeded in 24-well tissue culture dishes 24 hours before transfection. 20 ug luciferase plasmid and 10 ug Renilla plasmid were mixed into 2 mls serum-free DMEM. NFκB luciferase and AP-1 plasmids were purchased from BD Biosciences CLONTECH. pRLTK was used as an internal control for transfection efficiency.

300 ul Lipofectamine PLUS reagent (Invitrogen) was mixed with 200 ul serum-free DMEM. The PLUS reagent and plasmid were incubated for 15 minutes at room temperature. 100 ul Lipofectamine was added to 2.4 ml serum-free DMEM.

The lipofectamine and the DNA-PLUS solutions were mixed and incubated for an additional 15 minutes. The transfection mix was diluted into 20 mls of serum-free DMEM. 250 ul of diluted transfection mix was added to each well and the cells were incubated for 3 hours. Cells were lysed and the lysates assayed for luciferase activity.

A final stock concentration of 10 mM troglitazone (Cayman Chemical) was dissolved in DMSO and diluted to various concentrations. DMSO was added to the cells as a control. Cells were lysed in 1× Passive Lysis Buffer and assayed with the Dual Luciferase Reporter Assay System (Promega) on a microplate luminometer from Berthold Detection Systems.

Transcription Factor Arrays: TranSignal TF-TF Interaction Array I (Panomics) was processed according to the manufacturer's instructions. Nuclear extracts from 16HBEo-sense and antisense cells were incubated with biotin-labeled double-stranded oligonucleotides. PPARγ was immunoprecipitated with 3 μg of monoclonal antibody and Dynabeads (Dynal), which are magnetic protein G beads. Free cis-elements and non-specific binding proteins were washed away. PPARγ associated biotin-labeled probes were eluted from the beads and hybridized to TranSignal Protein/DNA array membranes. The arrays were blocked and incubated with Streptavidin-HRP and developed with a chemiluminescent detection system.

Immunoprecipitations: Nuclear and cytoplasmic extracts were prepared with the nuclear extraction kit (Panomics). Nuclear extracts were used for the immunoprecipitations with a polyclonal antibody against the NF-κB p50 subunit (Santa Cruz). The nuclear extract was diluted to 500 ul with immunoprecipitation buffer, 1% TX-100, 150 mM NaCl, 10 mM Tris pH 7.4, 1 mM EDTA and protease inhibitor cocktail (Sigma). Extracts were incubated with antibody and rotated 1 hour or overnight at 4° C. Antibody-antigen complexes were precipitated with Protein G beads (Roche). Beads were washed three times with cold IP buffer. Beads were eluted in SDS-PAGE sample buffer and boiled. The supernatant was run on 10% SDS-PAGE and transferred to nitrocellulose by electro blotting.

PPARγ was detected using the PPARγ western blot detection kit (Panomics). Blots were blocked in 3% nonfat dry milk in 1× Wash Buffer II and rocked overnight at 4° C. Affinity purified monoclonal:antibody (1:300) was incubated for 2 hours at room temperature. Blots were washed three times with 1× Wash Buffer II for 15 minutes. Anti-mouse HRP (1:1000) was added for 1 hour at room temperature. Blots were washed 4× with 1× Wash Buffer I for 20 minutes. The blots were developed using the Panomics chemiluminescent detection system.

### Results

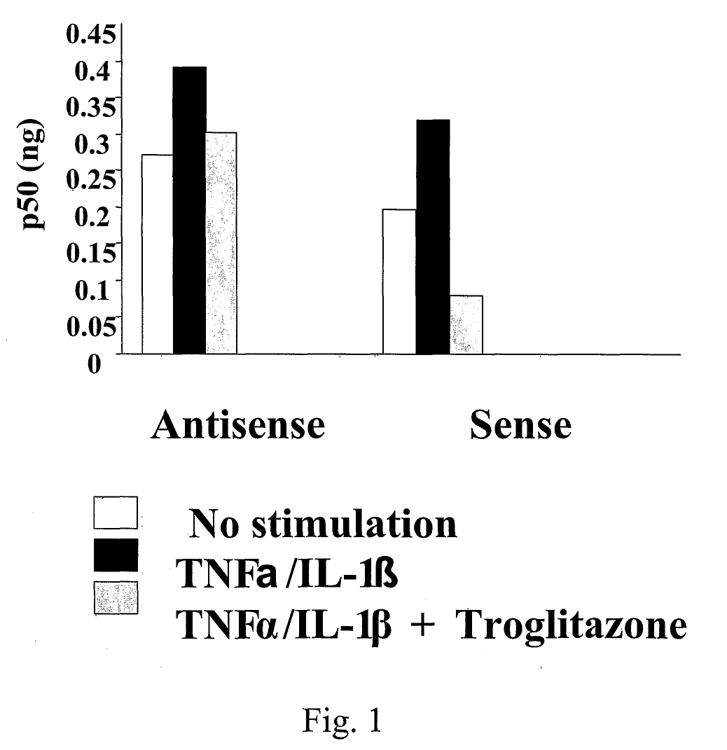

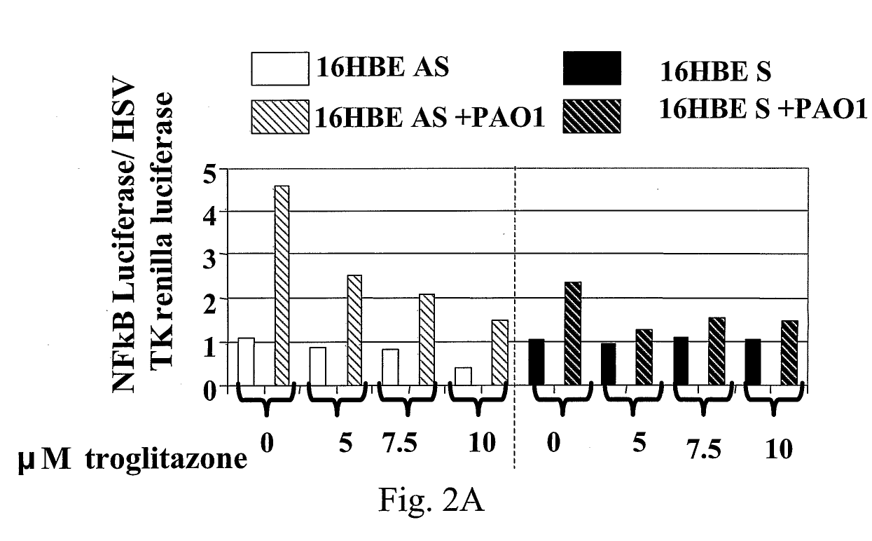

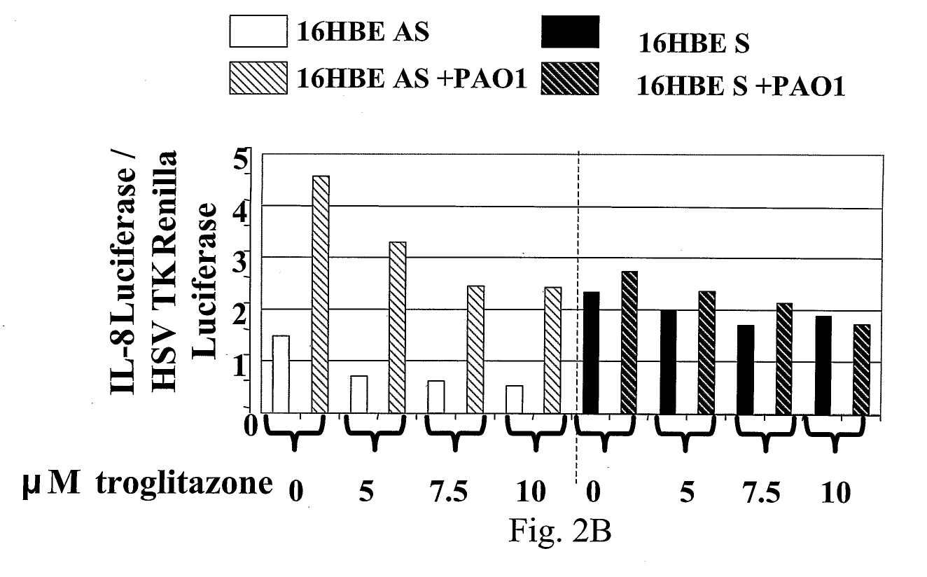

Confirmation that NF-κB is activated in excess in CF cell lines: Because one of our hypotheses is that failure of appropriate function of PPARγ contributes to the excess activation of NF-κB, we verified that NF-κB is activated in the CF cell lines in excess in two ways. In addition, to confirm the phenomenology in the cell lines we studied, we determined the amount of activated p50 in the nucleus of 16 HBEo-sense and antisense cells, under basal conditions, and under conditions of stimulation (FIG. 1). There was an increase in activated p50 in the nucleus in response to PAO1 in both sense and antisense cell lines and the amount was greater in the antisense (CF) cell lines than the sense (non-CF). In addition, we transfected into these cells constructs containing the luciferase gene driven by NF-κB elements, or the native IL-8 promoter. The cells were then exposed to PAO1 and promoter activity assessed by measuring luciferase activity. The antisense (CF phenotype) cell lines had greater luciferase expression in response to PAO1 than did the sense (nonCF phenotype) cells (FIG. 2). Thus, with three independent assays in two different model systems, the activation of NF-κB in CF models is in excess of that observed in non CF models.

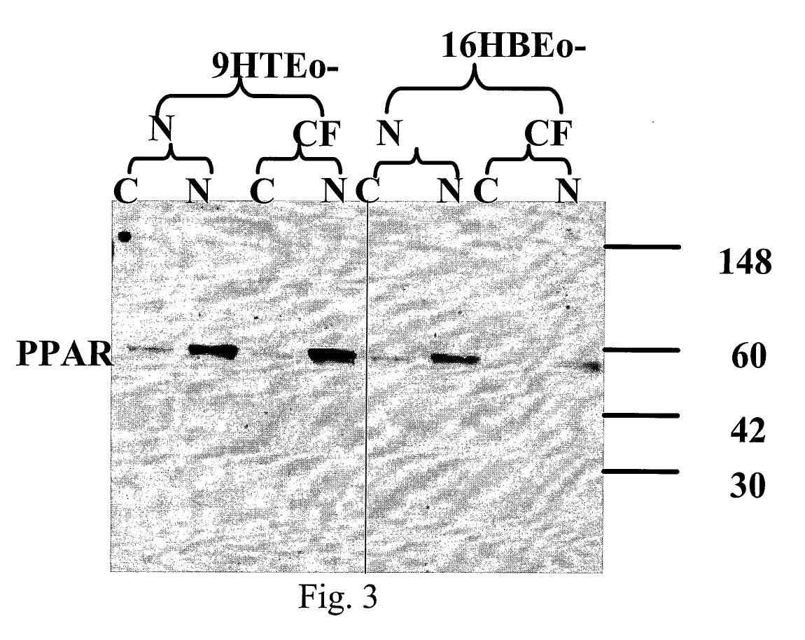

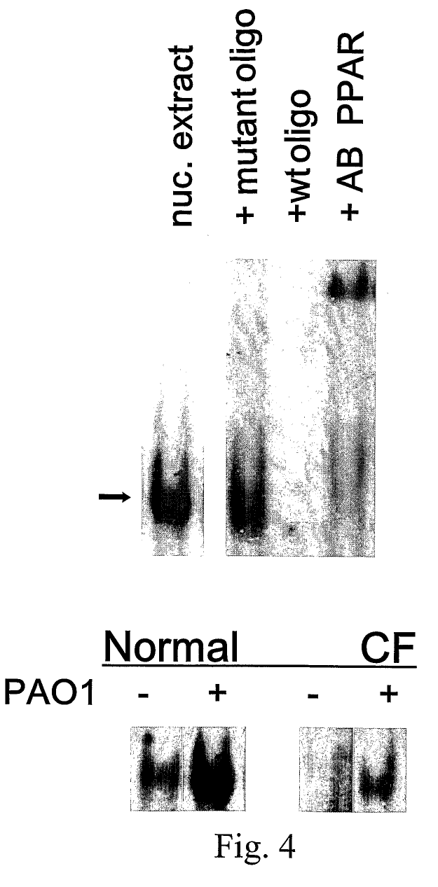

Identification of PPARγ in extracts of airway epithelial cells: Both cytoplasmic and nuclear extracts of 9HTEo- and 16HBEo-cell pairs (CF phenotype and non-CF phenotype) demonstrated the presence of PPARγ by Western blot (FIG. 3). In the 9HTEo-cell pairs, there appeared to be equivalent amounts of the protein in the CF and non-CF members of the pair. For the 16HBEo-cell pairs, however, the antisense (AS), or CF phenotype, member of the pair expressed less PPARγ than the sense congener (non-CF phenotype). This differential expression does not change if the cells are stimulated with PAO1 (FIG. 3B). EMSAs using the PPRE (FIG. 4) demonstrate DNA binding by components of the nuclear extract from these cell lines, which is markedly reduced by inclusion of cold probe, but not by cold probe of mismatched sequence, and which undergoes supershift with antibody to PPARγ, identifying the binding protein as PPARγ. For both the 9HTEo-pair and the 16HBEo-pair, the CF member of the pair displays less PPRE binding. Therefore, PPARγ is expressed in human airway epithelial cell lines, CF and non-CF, but appears to be less functional in binding its target DNA sequence in CF. Western blot confirms that PPARγ is also present in well-differentiated airway epithelial cells grown at the air-liquid interface (data not shown).

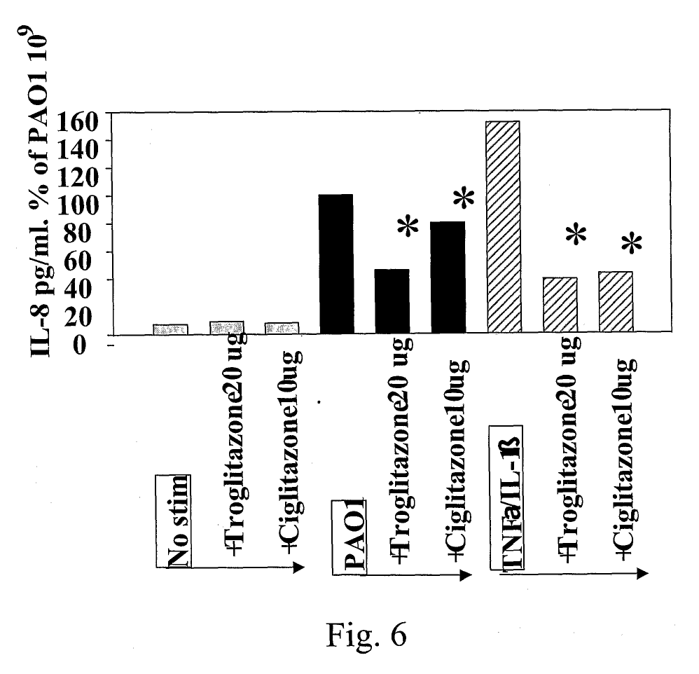

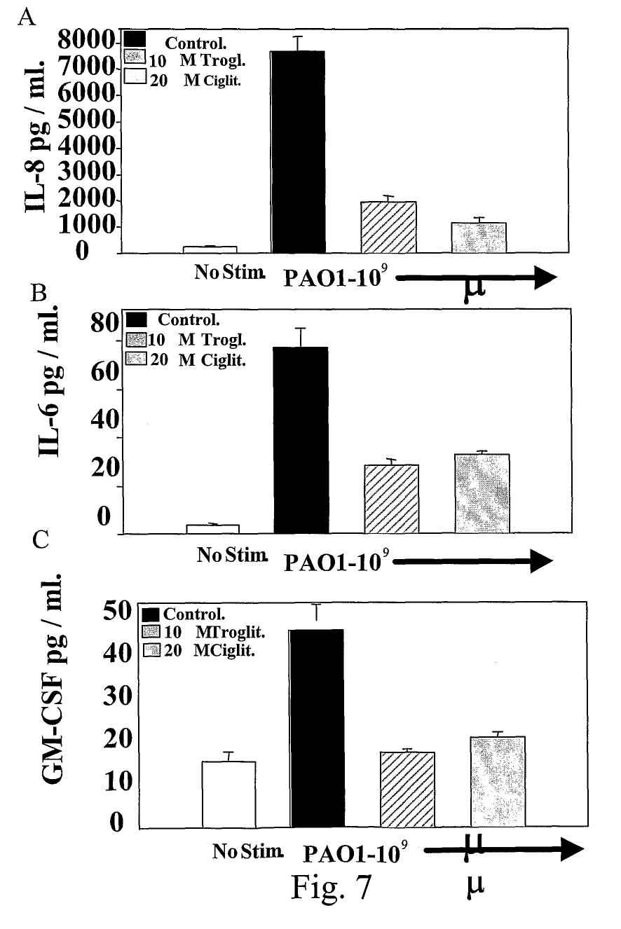

Cytokine and MMP-9 production by well-differentiated airway epithelial cells at the air-liquid interface is inhibited by agonists of PPARγ: When exposed at the apical surface to the laboratory strain of P. aeruginosa, PAO1 for one hour, or when stimulated by TNFα/IL-1β for one hour, well differentiated airway epithelial cells produced IL-8, IL-6, and GM-CSF in a dose-dependent fashion. The absolute amounts of cytokines produced varied from sample to sample, from different donors, but there was excellent agreement in the triplicate wells from a single donor. For all donors, there were measurable quantities of IL-8, but for cells from some donors, levels of IL-6, and/or GM-CSF were sometimes below the limits of detection. When PPAR agonists were added to the medium and cytokine production measured 6, 12 or 18 hr after stimulation, there was significant inhibition of cytokine production by the agonists (FIGS. 6 and 7). At or after 24 hours after stimulation, without replenishment of drug supply, inhibition was not evident (data not shown). Inhibition was dose dependent over the range of 0.1-10 mg/ml for troglitazone (data not shown).

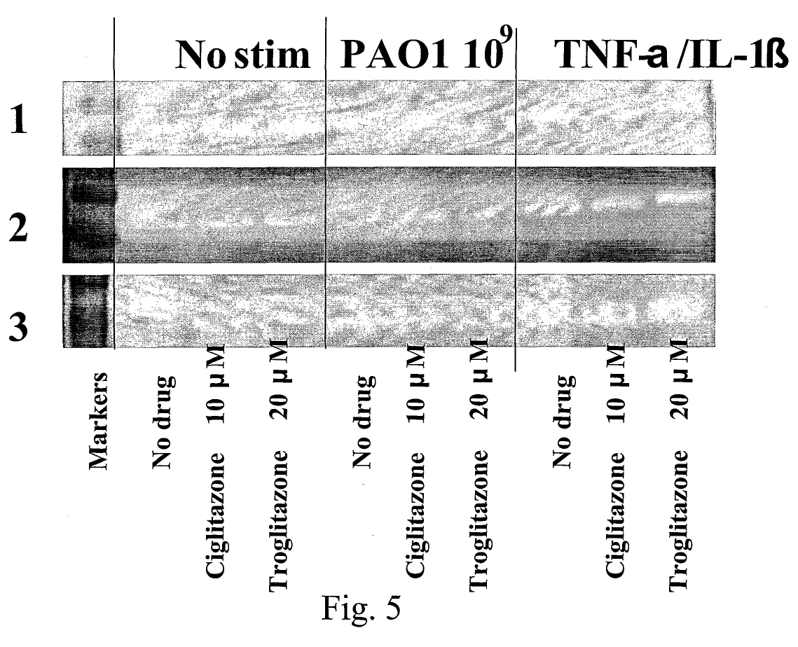

Gelatin zymography shows that well-differentiated airway epithelial cells grown at the air-liquid interface release MMP-9, which can digest the protein in the gel. Release of MMP-9 is also inhibited by PPARγ agonists (FIG. 5).

Activation of NF-κB is inhibited by agonists of PPARγ: 16HBEo-cell pairs transfected with a construct of NF-κB binding elements driving firefly luciferase displayed activation of luciferase activity after stimulation with PAO1. This activation was significantly inhibited by troglitazone, in dose-dependent fashion (FIG. 2). To test whether the NF-κB responsive elements would be affected by PPAR agonists in the context of a native promoter, we tested the effect of troglitazone on a luciferase construct driven by the upstream regulatory elements of the IL-8 gene. Similar inhibition was seen with PPAR agonists (FIG. 2). These transfections were not performed in the 9HTEo-pair because the two cell lines were very different in their ability to be transfected (the 9HTEo-pCEP R cell line expressed over 100 fold less reporter gene than the 9HTEo-pCEP cell line), making comparative studies difficult. They were not performed in the well-differentiated airway epithelial cells because these cells are very difficult to transfect.

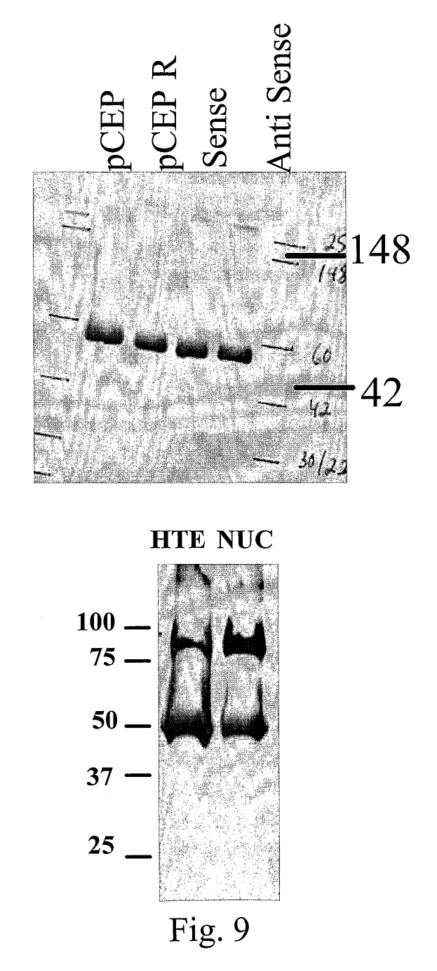

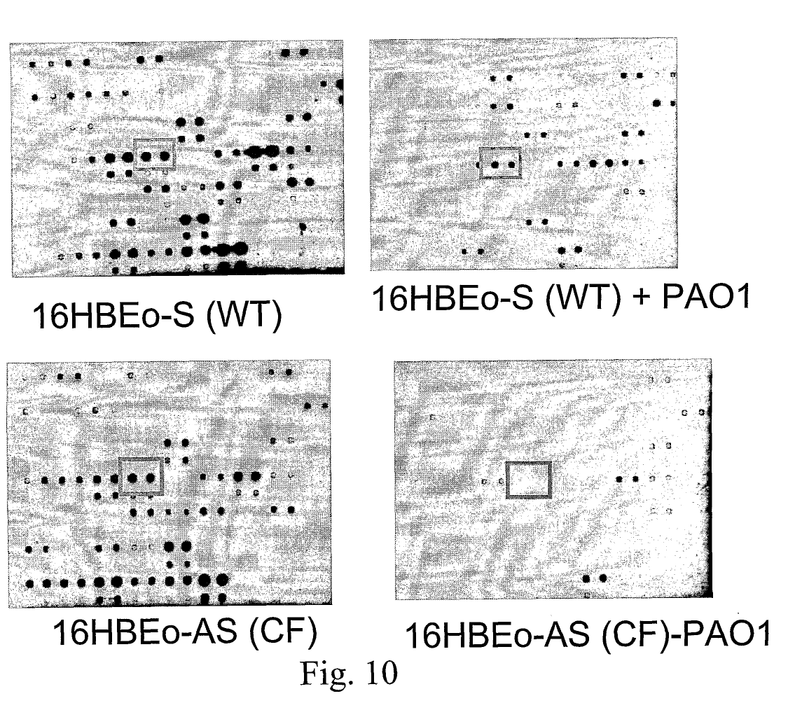

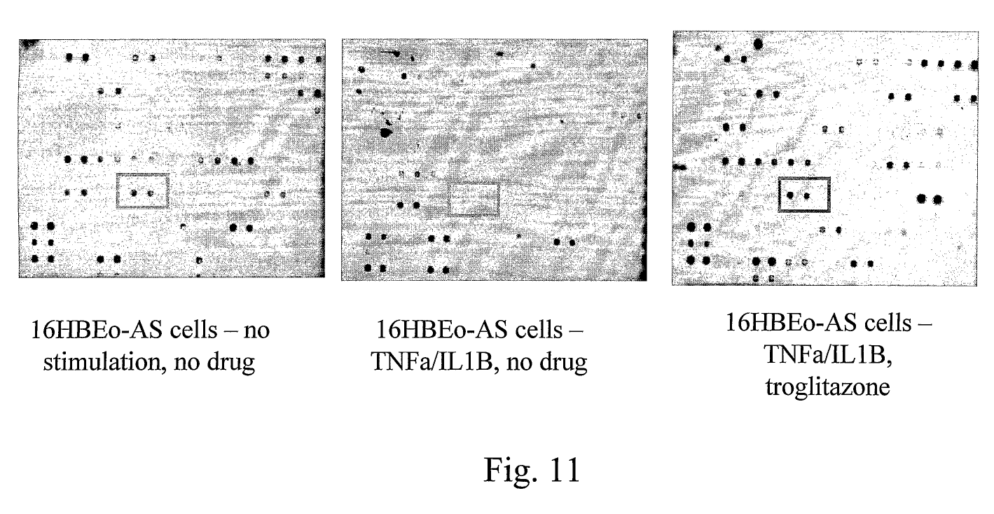

Interaction of NF-κB and PPARγ: In order to test whether PPARγ can interact directly with NF-κB, we conducted co-immunoprecipitation assays. Antibodies to both the p50 and the p65 subunits of NF-κB can pull down PPARγ (FIG. 8). In addition, antibodies to PPARγ also pulled down p65 and p50, though these assays had to be performed in whole cell extracts in order to recover sufficient PPARγ (FIG. 9). In a second, more sensitive assay, which capitalizes on the ability of the DNA target sequence of each transcription factor to bind specifically both to its cognate transcription factor and to its minus strand, interaction of PPARγ with NF-κB was also identified (FIGS. 10 and 11). This interaction was reduced by prior incubation of the cells with PAO1, but could be preserved in part by the inclusion of troglitazone in the incubation mix and in the subsequent culture media. These data suggest that although inflammatory stimulation causes changes in NF-κB that reduce its interaction with PPARγ, these changes can be partly abrogated by activation of PPARγ with an agonist ligand.

In order to test the impact of CFTR deficiency on the interaction of PPAR-γ with other transcription factors, we treated well differentiated airway epithelial cells grown at the ALI with I172 (20 uM), an inhibitor of CFTR activity, continuously for 72 hr prior to preparation of nuclear extracts or stimulation. This treatment has been shown to inhibit CFTR activity continuously by over 90%, as assessed by using chamber estimates of ion currents, without compromising cell viability. This model allows one to compare, in well-differentiated airway epithelial cells that have identical genetic endowment at all loci, the effect of CFTR inhibition on various cellular processes. Cells treated with I172 displayed less interaction between PPARγ and other transcription factors, particularly following stimulation with TNFα/IL1β. During the course of these experiments, cells from the airways of a patient with CF of genotype ΔF508/ΔF508, obtained at transplant, became available and were cultured at the air-liquid interface. These cells displayed vigorous stimulation of IL-8 in response to PAO1 or TNFα/IL-1β. Nuclear extracts of these cells showed very limited interaction between PPARγ and other transcription factors, including NF-κB. While this represents only a single sample, with the attendant possibility that variation at other genetic loci could produce the observed results, these results support the concept that in CF, reduced interaction of PPARγ and NF-κB may contribute to the excess activation of genes driven by NF-κB.

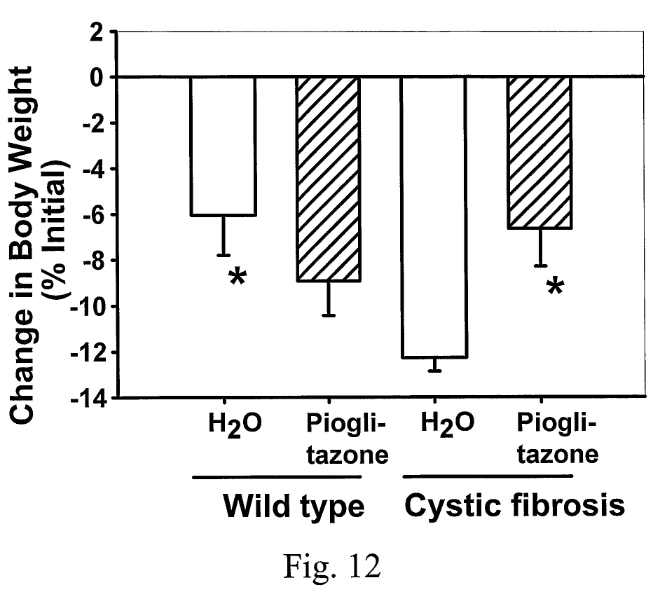

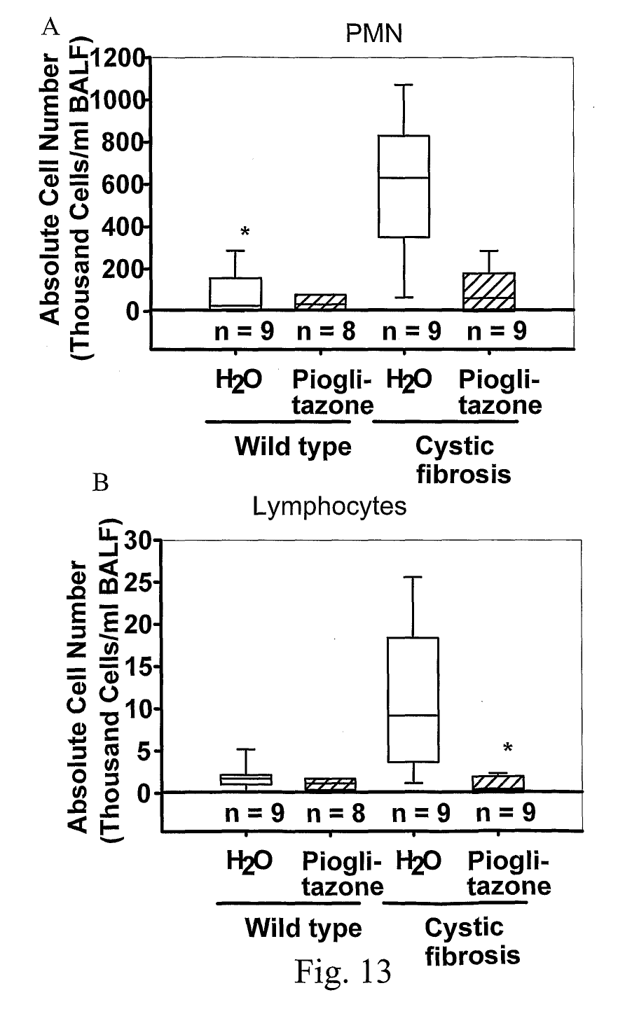

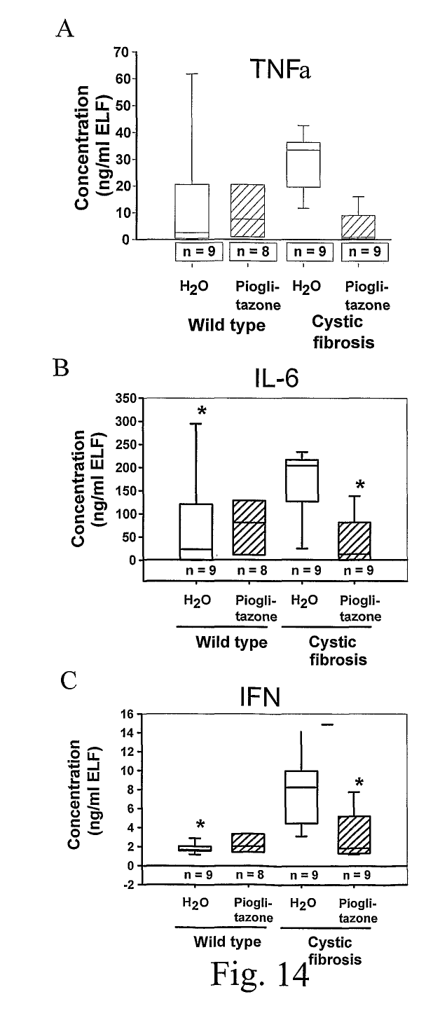

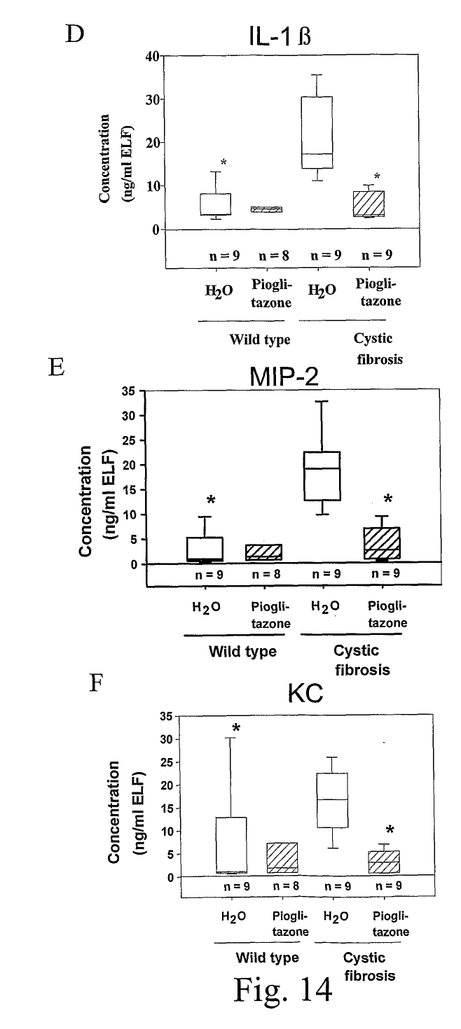

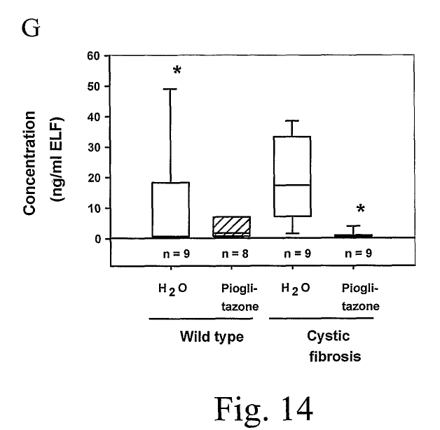

Pioglitazone inhibits the inflammatory response in CF mice to acute administration of Pseudomonas: Mice pretreated with pioglitazone or vehicle by gavage, then challenged with prior to challenge with M57-15 P. aeruginosa, underwent BAL for inflammatory response outcome measures 24 hours after challenge. Cell counts, cytokine values, and body weight were recorded. WT mice had similar inflammatory parameters and weight loss whether they received pioglitazone or vehicle. CF mice treated with vehicle had marked increase in inflammatory response compared to WT mice treated with vehicle, as previously reported for untreated mice (FIGS. 12-14). However CF mice treated with pioglitazone had significant reduction of the inflammatory response by pioglitazone.

PPARγ expression in airway epithelium of mice: Immunostaining for PPARγ is observed in airway epithelial cells in sections of mouse lung, whereas sections treated with the secondary antibody with no primary antibody show no signal. Expression is indistinguishable in airways from CF and WT mice, is present in both cytoplasm and nucleus, and does not change in intensity or location following acute infection with P. aeruginosa in either CF or WT mice, even in areas in which an inflammatory infiltrate is identified. Therefore, in contrast to findings described for intestinal epithelium, we cannot ascribe the differential anti-inflammatory response of CF and WT mice to pioglitazone to differences is subcellular localization of the protein following drug administration or infection.

### Discussion

In the lungs of CF children, exposure to bacteria results in neutrophil and IL-8 recruitment into the BAL fluid in excess of what is seen in infected non-CF control young children, even when controlled for burden of organisms in the lungs. Some studies in CF infants suggest that inflammation may even precede infection, though it is difficult to exclude the possibility that those infants were infected earlier and the inflammatory response simply persists well after the infectious agents can no longer be detected. CF mice of various genotypes (G551D, S489X, ΔF508, Y122X, R117H) on different genetic backgrounds (CD-1, C57BL/6, mixed C57BL/6 and 129, and mixed C57BL/6, 129, and FV/B) studied in at least three different laboratories around the world, challenged with pseudomonas embedded in agarose beads, have excess cytokines and inflammatory cells in BAL fluid. In addition, in response to acute challenge with pseudomonas, CF mice have greater cell and cytokine response, even though they kill the bacteria at least as well as their wild type counterparts. This inflammatory response is itself an independent contributor to the progression of the CF lung disease, because when inflammation is inhibited by alternate-day steroids or high dose ibuprofen, the rate of decline of pulmonary function is slowed. However, adverse effects from alternate-day steroids are prohibitive in CF, and the increased incidence of the rare complication of gastrointestinal hemorrhage with high dose ibuprofen has made many clinicians avoid its use, despite unequivocal evidence of benefit. Understanding and controlling the inflammatory response without harming the host defenses against bacteria and without incurring adverse effects could be of great benefit to CF patients.

Most, but not all, published data suggest that airway epithelial cells may contribute to the excess inflammatory response in CF. These cells are good candidates to contribute to the inflammatory response because they are the initial site of contact with the outside world and often the first cells to contact inhaled bacteria, they are known to express CFTR and to manifest its lack by altered salt transport and other abnormalities, such as reduced NOS-2 expression, and CF mice whose airway epithelial cells have been corrected by expression of the CFTR transgene driven by the K18 promoter only in epithelial cells only lack the excess inflammation in response to agarose containing agar beads. Human airway epithelial cells in culture with the CF phenotype usually, but not invariably, produce more IL-8 and sometimes other cytokines in response to PAO1 or its products, or TNF-α and IL-1β. Data from several laboratories indicate that activation of NF-κB occurs in excess in CF airway epithelial cells. Increased NF-κB driven transcription could account for the increased IL-8, IL-6, GM-CSF, ICAM-1 and other inflammatory proteins that have been detected in the surface or media from CF airway epithelial cells. Our data in our well-matched cell lines and in WD AECs treated or not with the CFTR inhibitor, I172, confirm the reports of increased IL-8, IL-6, and/or GM-CSF in CF phenotype cells in response to PAO1 or TNF-α plus IL-1β. Our data also indicates that increased activation of NF-κB is associated with this increase in proinflammatory mediator production in both cell lines and well differentiated cells grown at the air-liquid interface.

The nuclear receptor, PPARγ, is expressed in airway epithelial cells. When PPARγ ligands are administered along with or prior to inflammatory stimuli, NF-κB driven processes are inhibited, including the production of IL-8, IL-6, and GM-CSF and the release of matrix metalloproteinase 9 (MMP9) in response to pseudomonas or cytokine stimulation. Transcription from an NF-κB luciferase construct or one in which the IL-8 promoter is used to drive luciferase is reduced by agonists of PPARγ in airway epithelial cells, indicating that these agonists may exert at least a portion of their activity at the level of gene transcription. Here we show that the mechanism by which this occurs is could be by direct interaction with NF-κB or by interaction with a third protein, possibly a DNA helicase, to which both NF-κB and PPARγ bind. Both the p50 and the p65 subunits co-immunoprecipitated with PPARγ, and PPARγ co-immunoprecipitated with specific antibodies to both p50 and p65. This interaction was confirmed by another technique of recognizing interaction, which is much more sensitive because it recognizes transcription factors by DNA base pairing in their target sequences. These data indicate that the failure of interaction between NF-κB and PPARγ in CF, especially under conditions of inflammatory stimulation, is mirrored by the reduced interactions of PPARγ with many other transcription factors, some of which also drive or promote inflammatory processes. Thus, the interactions of PPARγ with transcription factors could have broad implications for regulation of the inflammatory process. PPARγ interacts with AP-1 and AP-2, which are required for transcription of MMP-9. Other transcription factors identified in these arrays are: RXR, the known binding partner of PPARγ as well as Stat 1 and Stat 4. The interaction of PPARγ with these other transcription factors, including AP-1 and AP-2, is also attenuated when the cells are stimulated with PAO1 or TNFα/IL-1β, and the attenuation is rescued by troglitazone, both in the CF and the non-CF cell lines. The specific mechanisms by which PPAR Y interaction is reduced by proinflammatory stimuli are not clear. However, the most parsimonious explanation for the near-universal concomitant decrease in interaction of PPARγ with other transcription factors as well as the rescue of interaction with nearly all these factors by troglitazone is that a conformational change has taken place in PPARγ in the face of inflammatory stimuli, probably by post-translational modification (e.g., by phosphorylation) which reduces its ability to interact with other transcription factors. Binding to troglitazone could then either protect PPARγ from the post-translational modification, or change its conformation so that, even if it is modified, it can still interact with transcription factors. It seems unlikely that concomitant changes take place in all the other transcription factors in the array that alter their ability to interact with PPARγ. However, it is possible that the inflammatory process alters a common binding partner of all the transcription factors, such as a helicase, and it is this change, rather than changes in PPARγ, that alters the interactions we observe. However, the fact that we also observe that inflammatory stimulation increases binding of PPARγ to its target DNA sequence in the EMSA assay, together with the ELISA data indicating increased PPRE binding of PPARγ in nuclear extracts following inflammatory stimulation suggests that there the conformational changes in PPAR that reduce its ability to interact with other transcription factors, may increase its propensity to bind to its DNA target sequence. One possible explanation is that activation of kinases such as JNK and ERK following inflammatory stimuli can phosphorylate PPARγ in such a way as to promote its inactivation and degradation. If PPARγ is bound to its ligand, it may remain in a conformation less favorable for phosphorylation and subsequent accelerated degradation.

The DNA binding activity of PPARγ is reduced in CF airway epithelial cells. EMSAs indicate less interaction of PPARγ with its target DNA sequence in two CF model systems compared to matched controls. For the 16HBEo-cells, this could be due, at least in part, to reduced expression of PPARγ in the CF member of the pair, as demonstrated by Western blot, but in the 9HTEo-cell pair, expression is comparable in the CF and the non-CF members of the pair. It seems most likely that the ability of PPARγ to bind to its target DNA sequence is reduced. The 9HTEo-cell pair differs from the 16HBEo-cell pair in that the 16HBEo-pair displays activation of IL-8 and IL-6 production at baseline, but the 9HTEo-cell lines are quiescent until a stimulus is applied, and the basal production of cytokines is minimal. If the continuous activation in the 16HBEo-cells results in more rapid degradation of PPARγ, this might account for the greater deficit in CF cells in this cell line. It is possible that the CF cell lines exist in a heightened inflammatory state and PPARγ is sensitive to this constitutive activation. In this CP mouse model, application of troglitazone results in the proper nuclear translocation of the PPARγ in the gut, which is not observed in the absence of ligand. However, we did not observe these changes in localization of PPAR immunostaining in the lungs of CF knockout mice compared to wild type. It may be that in the lung, the expression of PPARγ is quite sensitive to the inflammatory environment, and any changes we observe in CF may be due to heightened inflammatory tendencies. This suggestion is supported by the reduction of PPARγ in patients with asthma or alveolar proteinosis, diseases characterized by inflammation, but normal CFTR. Even if the changes in PPARγ are associated with changes in the inflammatory milieu in CF and are not related to the CF defect itself, they could in turn contribute to the excess inflammatory response and could represent a valid therapeutic target. It now appears that some, but not all, nonsteroidal anti-inflammatory drugs (NSAIDs) can ligate PPARγ. Ibuprofen, at the concentration required to observe the therapeutic effect in CF, is one of those drugs. Ligation of PPARγ might, therefore, be the mechanism of action of one of the proven anti-inflammatory therapeutic agents in CF.

In order to test the therapeutic potential of PPARγ agonists in CF animals, we utilized the acute pseudomonas challenge model in CF and non-CF mice, because it was the closest mimic of the acute pseudomonas challenge applied to the epithelial cells in culture. We administered pioglitazone by gavage because this is one of the two PPARγ agonists available for human use. In two of the three experiments, pioglitazone limited the inflammatory response in the CF mice. However, the dose used in these studies was high compared to conventional human doses, on a weight basis, and the drug was administered prior to challenge, a luxury that may not be available for many patients with CF.

1. A method of treating a subject with a cystic fibrosis related disorder comprising:
administering at least one PPARγ agonist or a derivative thereof to cystic fibrosis cells of the subject in an amount effective to inhibit NF-κB activation in the cystic fibrosis cells, the PPARγ agonist or the derivative thereof comprising a thiazolidinedione or a derivative thereoaf.

2. The method of claim 1, the amount of the PPARγ agonist or derivative thereof being administered to the subject being that amount effective to suppress airway inflammation.

3. (canceled)

4. (canceled)

5. (canceled)

6. (canceled)

7. The method of claim 1, the PPARγ agonist or a derivative thereof comprising a compound of Formula IV or pharmaceutically acceptable salt of a compound of Formula IV, wherein Formula IV is: 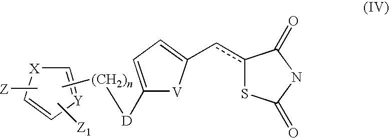
wherein the dotted line represents a bond or no bond; V is HCH—, —NCH—, —CH═N—, or S;
D is CH₂, CHOH, CO, C═NOR₁₇, or CH═CH; X is S, SO, NR₁₈, —CH—N, or —N═CH;
Y is CH or N;
Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo;
Z, is hydrogen or (C₁-C₃)alkyl;
R₁₇ and R₁₈ are each independently hydrogen or methyl; and n is 1, 2, or 3.

8. The method of claim 1, the PPARγ agonist or a derivative thereof comprising a compound of Formula V or pharmaceutically acceptable salt of a compound of Formula V, wherein Formula V is: 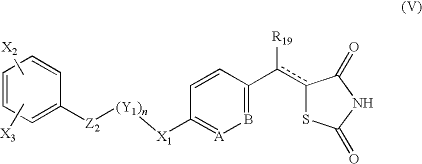
wherein the dotted line represents a bond or no bond;
A and B are each independently CH or N with the proviso that when A or B is N the other is CH; X is S, SO, SO₂, C₁₋₂, CHOH, or CO;
n is O or 1;
Y₁ is CHR₂₀ or R₂₁, with the proviso that when n is I and Y, is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂;
R₁₉, R₂₀, R₂₁, and R₂₂ are each independently hydrogen or methyl; and
X₂ and X₃ are each independently hydrogen, methyl, trifluoromethyl, phenyl, benzyl, hydroxy, methoxy, phenoxy, benzyloxy, bromo, chloro, or fluoro.

9. The method of claim 1, the PPARγ agonist or a derivative thereof comprising a compound of Formula II or pharmaceutically acceptable salt of a compound of Formula VI, wherein Formula VI is: 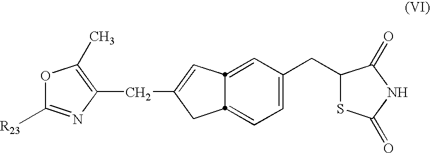
wherein R₂₃ is alkyl of 1 to 6 carbon atoms, cycloalkyl of 3 to 7 carbon atoms, phenyl or mono- or all-substituted phenyl wherein the substituents are independently alkyl of 1 to 6 carbon atoms, alkoxy of 1 to 3 carbon atoms, halogen, or trifluoromethyl.

10. The method of claim 1, the PPARγ agonist or a derivative thereof comprising a compound of Formula VII or pharmaceutically acceptable salt of a compound of Formula VII, wherein Formula VII is: 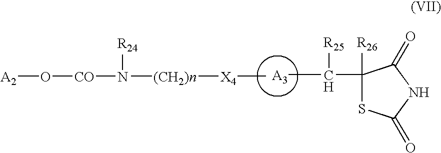
wherein A² represents an alkyl group, a substituted or unsubstituted aryl group, or an aralkyl group wherein the alkylene or the aryl moiety is substituted or unsubstituted;
A³ represents a benzene ring having in total up to 3 optional substituents;
R₂₄ represents a hydrogen atom, an alkyl group, an acyl group, an aralkyl group wherein the alkyl or the aryl moiety is substituted or unsubstituted, or a substituted or unsubstituted aryl group; or
A² together with R₂₄ represents substituted or unsubstituted C₂₋₃ polymethylene group;
R₂₅ and R₂₆ each represent hydrogen, or R₂₅ and R₂₆ together represent a bond; X₄ represents O or S; and
n represents an integer in the range from 2 to 6.

11. The method of claim 1, the PPARγ agonist or a derivative thereof comprising a compound of Formula VIII or pharmaceutically acceptable salt of a compound of Formula VIII, wherein Formula VIII is: 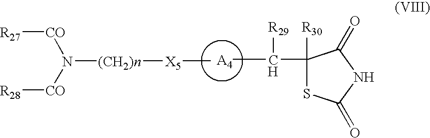
wherein: R₂₇ and R₂₈ each independently represent an alkyl group, a substituted or unsubstituted aryl group, or an aralkyl group being substituted or unsubstituted in the aryl or alkyl moiety;
or R₂₇ together with R₂₈ represents a linking group, the linking group consisting or an optionally substituted methylene group or an O or S atom; R₂₉ and R₃₀ each represent hydrogen, or R₂₉ and R₃₀ together represent a bond;
A₄ represents a benzene ring having in total up to 3 optional substituents;
X₅ represents O or S; and
n represents an integer in the range of 2 to 6.

12. The method of claim 1, the PPARγ agonist or a derivative thereof comprising a compound of Formula IX or pharmaceutically acceptable salt of a compound of Formula IX, wherein Formula IX is: 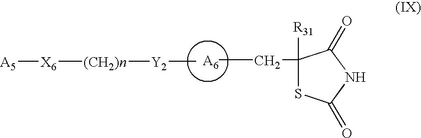
wherein: A₅ represents a substituted or unsubstituted aromatic heterocyclyl group; A₆ represents a benzene ring having in total up to 5 substituents;
X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group;
Y₂ represents O or S;
R₃₁ represents an alkyl, aralkyl, or aryl group; and n represents an integer in the range from 2 to 6.

13. The method of claim 1, the PPARγ agonist or a derivative thereof comprising a compound of Formula X or pharmaceutically acceptable salt of a compound of Formula X, wherein Formula X is: 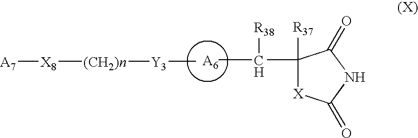
wherein; A₇ represents a substituted or unsubstituted aryl group;
A₈ represents a benzene ring having in total up to 5 substituents;
X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group;
Y₃ represents O or S;
R₃₇ represents hydrogen;
R₃₈ represents hydrogen or an alkyl, aralkyl, or aryl group or R₃₇ together with R₃₈ represents a bond; and
n represents an integer in the range from 2 to 6.

14. (canceled)

15. (canceled)

16. The method of claim 1, the PPARγ agonist or a derivative thereof comprising at least one compound or a pharmaceutically salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxy]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione, 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidie-2,4-dione, 5-[[4-(3-hydroxy-1-methylcyclohexyl)methoxy]benzyl]thiazolidine-2,4-dione; 5-[4-[2-(5-methyl-2-phenyloxazol-4-yl)ethoxyl)benzyl]thiazolidine-2,4-dione; 5-[(2-benzyl-2,3-dihydrobenzopyran)-5-ylmethyl]thiazolidine-2,4-dione; 5-[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-2[-(N-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

17. A method of treating inflammation associated with NF-κB activation in a subject the method comprising:
administering a therapeutically effective amount of at least one PPARγ agonist or a derivative thereof to cells expressing NF-κB in the subject effective to inhibit NF-κB activation of the cells, the PPARγ agonist or a derivative thereof comprising at least one compound or a pharmaceutically salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl)-5-methlthiazolidine-2,4-dione, 5-(4-[2-[2,4-dioxo-5-phenylthiazolidine-3-yl)ethoxy]benzyl]thiazolidine-2,4-dione; 5-[4-(2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidin-2,4-dione; 5-[4-[2-(3-phenylureido)ethoxyl]benzylthiazolidine-2,4-dione; 5-[4-[2-(N-benzoxazol-2-yl)-N-metholamino]ethoxy]benzyl]thiazolidine-2,4-dione; 5-[[3-(5-methyl-2-phenyloxazol-4-yl)propionyl]benzyl]thiazolidine-2,4-dione; 5-(2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

18. The method of claim 17, the inflammation being associated with a cystic fibrosis related disorder.

19. (canceled)

20. (canceled)
